(19) United States
(12) Patent Application Publication
(10) Pub. No.: US 2023/0398208 A1
(43) Pub. Date: Dec. 14, 2023

(54) SARS-CoV-2 VACCINES AND USES THEREOF

(71) Applicant: FRED HUTCHINSON CANCER CENTER, Seattle, WA (US)

(72) Inventors: Jason Price, Seattle, WA (US); James Olson, Seattle, WA (US); Colin E. Correnti, Seattle, WA (US); Barry L. Stoddard, Seattle, WA (US)

(73) Assignee: FRED HUTCHINSON CANCER CENTER, Seattle, WA (US)

(21) Appl. No.: 18/331,879

(22) Filed: Jun. 8, 2023

Related U.S. Application Data
(60) Provisional application No. 63/366,058, filed on Jun. 8, 2022.

(51) Int. Cl. A61K 39/215; A61K 39/39; A61K 45/06

(52) U.S. Cl. CPC A61K 39/215; A61K 39/39; A61K 45/06; A61K 2039/55505

(57) ABSTRACT

SARS-CoV-2 vaccines are described herein. The SARS-CoV-2 vaccine antigens can be presented on a circular tandem repeat protein (cTRP) scaffold as trimers or tetramers. The vaccines can be used to treat and/or reduce the risk of SARS-CoV-2 infection and to treat and/or reduce the risk of side effects associated with SARS-CoV-2 infection, such as SARS-CoV-2-related symptoms, syndromes, and complications.

### CROSS REFERENCE TO RELATED APPLICATION

This application claims priority to U.S. Provisional Patent Application No. 63/366,058 filed Jun. 8, 2022, the contents of which are incorporated by reference as if fully set forth herein.

### REFERENCE TO SEQUENCE LISTING

The Sequence Listing associated with this application is provided in text format in lieu of a paper copy, and is hereby incorporated by reference into the specification. The name of the text file containing the Sequence Listing is 2WP4683_ST26.txt. The text is 272 KB, was created on Jun. 6, 2023, and is being submitted electronically via Patent Center.

### FIELD OF THE DISCLOSURE

SARS-CoV-2 vaccines and uses of the same are described. The SARS-CoV-2 vaccine antigens can be presented as trimers or tetramers on a circular tandem repeat protein (cTRP) scaffold, and can be used to treat and/or reduce the risk of SARS-CoV-2 infection and also to treat and/or reduce the risk of complications associated with SARS-CoV-2 infection.

### BACKGROUND OF THE DISCLOSURE

SARS-CoV-2, the viral causative agent of COVID-19, has infected over 191 million people since it emerged in 2019, killing more than 4 million. Despite the great promise of vaccines developed to date, the resulting pandemic is ongoing. Waning immunity, the biological and behavioral diversity of the human population, and the emergence of viral variants that challenge monoclonal therapies and vaccine efficacy indicate that future outbreaks are highly likely (Diamond et al., 2021, Res Sq. doi: 10.21203/rs.3.rs-228079/v1; Fraser et al., 2004, Proc Nati Acad Sci USA 101, 6146-6151; Lavine et al., 2021, Science 371, 741-745; Wang et al., 2021, doi: 10.1101/2021.01.25.428137; Wang et al., 2021, Nature 592(7855):616-622). As such, multi-pronged containment strategies will be required for many years to keep SARS-CoV-2, future variants and novel coronaviruses at bay (McKenna, 2021, Scientific American 324; Phillips, 2021, Nature 590, 382-384; Steenhuysen and Kelland, 2021, In Reuters (New York: Thomson Reuters); Weisblum et al., 2020, Elife 9).

### SUMMARY OF THE DISCLOSURE

The current disclosure provides SARS-CoV-2 vaccines and uses of the same. The SARS-CoV-2 vaccine antigens can be presented as trimers or tetramers on a circular tandem repeat protein (cTRP) scaffold, and can be used to treat and/or reduce the risk of SARS-CoV-2 infection and also to treat and/or reduce the risk of symptoms, syndromes, and complications associated with SARS-CoV-2 infection.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. Depiction of a 15 Å foldon, an 18 Å trimer tag, and a 54 Å circular tandem repeat protein (cTRP).

FIG. 2. SARS-CoV-2 Spike—cTRP: Model of the SARS-CoV-2 Spike glycoprotein (referred to herein as Spike or S protein) trimer scaffolded with a cTRP trimer. Top left: Side view of cTRP (20 Å) and spike glycoprotein (140 Å); bottom left: bottom view showing cTRP base (100 Å), top right: top view of spike glycoprotein on cTRP base (120 Å from point-to-point); and bottom right: perspective view of SARS-CoV-2 cTRP vaccine disclosed herein.

FIGS. 3A, 3B. SARS-CoV-2 vaccine sequences and subcomponents thereof. (3A) SARSCoV2_Spike_2Pmut-GGGS(4)-cTRP24(8)SS-TEV-HisAvi and subcomponents thereof. SARSCoV2_Spike_2Pmut-GGGS(4)-cTRP24(8)SS self-asembles to provide trimeric vaccine antigen presentation. (3B) SARSCoV2_Spike_RBD-cTRP24(6)SS-TEV-HisAvi and subcomponents thereof. SARSCoV2_Spike_RBD-cTRP24(6)SS self-assembles to provide tetrameric vaccine antigen presentation.

FIG. 4. Production of spike-cTRP (cTRPs can also be referred to as a toroid) and spike-foldon constructs in HEK293F cells: Spike expression and stability is improved with cTRP scaffold vs foldon comparator.

FIG. 5. Biochemical characterization of spike-cTRP construct Spike-cTRP and spike-foldon constructs bind to ACE2 in solution and on the cell surface.

FIG. 6. Neutralizing antibody titer-Mice immunized with spike-cTRP had higher neutralizing antibody titer than comparator spike-foldon immunized mice.

FIG. 7. Depiction of cTRP presenting different SARS-CoV-2 vaccine antigens on the “top” and “bottom” of the cTRP.

FIG. 8. Additional sequences supporting the disclosure including exemplary cTRP scaffolds creating alpha helices and circular and closed (also referred to as “stapled”) architectures. The following nomenclature can be used to reference specific cTRPs: “dTor (number of structural repeats)×(number of amino acid residues within each structural repeat)(protein handedness)”. For example, “dTor_3x33L” describes a cTRP protein including a single protein chain containing 3 repeats of 33 repetitive amino acids, wherein the helical bundles of that cTRP are entirely left-handed. In the case that a cTRP is assembled from multiple protein subunits that each contain a fraction of the total repeats in that cTRP, the nomenclature also may indicate the number of repeats in each subunit of a multimeric assemblage, and thereby can distinguish between multimeric cTRPs that contain the same total number of repeats. For example, “dTor_12x31L_sub3” describes an assembly of 4 identical protein subunits that each have 3 left-handed repeats, that come together to create a multimeric cTRP with 12 total repeats. In contrast, “dTor_12x31L_sub4” describes an assembly of 3 identical protein subunits that each have 4 repeats, that again come together to create a multimeric cTRP with 12 total repeats. Within FIG. 8, Group I provides sequences that form alpha-helical segments with N-terminal or C-terminal mutation positions to form a stapled cTRP bolded and underlined (sequences with no potential mutation positions or no naturally occurring cysteines at position 1 or 3 are not included in the 1st alpha helix of N-terminal or C-terminal segments (SEQ ID NOs: 59, 139, 84, 140, 85-88, 58, 89, 17, 90, 60-63, and 18-20); FIG. 8 Group II provides thick cTRP sequences that form alpha-helical segments with N-terminal or C-terminal mutation n positions at 5 and 7 bolded and underlined (SEQ ID NOs: 91-93, 80, 81, and 94); FIG. 8 Group Ill provides modified N-terminal segments (SEQ ID NOs: 95-101); FIG. 8 Group IV provides modified C-terminal segments (SEQ ID NOs: 89, 99, 103-105); FIG. 8 Group V provides sequences with N-terminal segment mutation positions and/or C-terminal segment mutation positions underlined and bolded with leading linker sequences (SEQ ID NOs: 107-110); FIG. 8 Group VI provides sequences with N-terminal segment mutations with leading linker sequences (SEQ ID NOs: 111-114, 115, 116, 133, and 118); FIG. 8 Group VII provides sequences with C-terminal segment mutations with leading linker sequences (SEQ ID NOs: 119-123); FIG. 8 Group VIII provides sequences with N-terminal segment mutation positions and/or C-terminal segment mutation positions underlined and bolded with leading and following linker sequences (SEQ ID NOs: 124-127); FIG. 8 Group IX provides sequences with N-terminal segment cysteine mutations with leading and following linker sequences (SEQ ID NOs: 128-135); FIG. 8 Group X provides sequences with C-terminal segment cysteine mutations with leading and following linker sequences (SEQ ID NOs: 136-138); FIG. 8 Group XI provides sequences with N-terminal segment mutation positions and/or C-terminal segment mutation positions underlined and bolded with leading and following linker sequences as well as secondary alpha helix forming sequence (SEQ ID NOs: 75 and 76-78, 139, 21-23, 64-68); FIG. 8 Group XII provides sequences with N-terminal segment cysteine mutations underlined and bolded with leading and following linker sequences as well as secondary alpha helix forming sequence (SEQ ID NOs: 140-152); FIG. 8 Group XIII provides sequences with C-terminal segment cysteine mutations to form a stapled cTRP underlined and bolded with leading and following linker sequences as well as secondary alpha helix forming sequence (SEQ ID NOs: 153-162); single chain cTRP with 24 repeats.

### DETAILED DESCRIPTION

SARS-CoV-2, the viral causative agent of COVID-19, has infected over 191 million people since it emerged in 2019, killing more than 4 million. Despite the great promise of vaccines developed to date, the resulting pandemic is ongoing. Waning immunity, the biological and behavioral diversity of the human population, and the emergence of viral variants that challenge monoclonal therapies and vaccine efficacy indicate that future outbreaks are highly likely (Diamond et al., 2021, Res Sq. doi: 10.21203/rs.3.rs-228079/v1; Fraser et al., 2004, Proc Nati Acad Sci USA 101, 6146-6151; Lavine et al., 2021, Science 371, 741-745; Wang et al., 2021, doi: 10.1101/2021.01.25.428137; Wang et al., 2021, Nature 592(7855):616-622). As such, multi-pronged containment strategies will be required for many years to keep SARS-CoV-2, future variants and novel coronaviruses at bay (McKenna, 2021, Scientific American 324; Phillips, 2021, Nature 590, 382-384; Steenhuysen and Kelland, 2021, In Reuters (New York: Thomson Reuters); Weisblum et al., 2020, Elife 9).

The current disclosure provides SARS-CoV-2 vaccines. The disclosed SARS-CoV-2 vaccines include a SARS-CoV-2 antigen presented on a circular tandem repeat protein (cTRP) scaffold. cTRPs generally include 24 repeats of a computationally designed alpha-helical sequence that self-assembles into a toroidal structure that can be utilized to present/scaffold proteins with selected valency. cTRPs with 12 repeats form toroidal dimers, cTRPs with 8 repeats form toroidal trimers, cTRPs with 6 repeats form toroidal tetramers, and cTRPs with 4 repeats form toroidal hexamers, allowing for the presentation/scaffolding of dimeric, trimeric, tetrameric, and hexameric proteins.

In particular embodiments, the SARS-CoV-2 vaccines disclosed herein utilize cTRP scaffolding to present SARS-CoV-2 spike glycoproteins or fragments thereof as trimers or tetramers. Trimeric presentation provides the unique ability to coordinate and stabilize the Spike prefusion state, as well as providing can improve immunogenicity, expression, and stability. Tetrameric presentation similarly can improve immunogenicity, expression, and stability of the disclosed vaccines.

SARS-CoV-2 vaccines disclosed herein can be utilized as subunit protein vaccines and/or as DNA/mRNA administered vaccines. The designs may also be used as immunogens for subsequent isolation of binding/neutralizing antibodies against the presented glycoproteins with therapeutic/diagnostic/research applications.

In particular embodiments, SARS-CoV-2 vaccines disclosed herein can be formulated to treat a SARS-CoV-2-infected subject or a subject at risk of SARS-CoV-2 infection. In particular embodiments, SARS-CoV-2 vaccines reduce the risk or severity of SARS-CoV-2 infection, and/or induce an immune response against SARS-CoV-2. Treating SARS-CoV-2 can reduce SARS-CoV-2 infection and/or treat a condition associated with SARS-CoV-2 infection, such as fever, severe headache, muscle pain, malaise, extreme asthenia, conjunctivitis, popular rash, dysphagia, nausea, vomiting, bloody diarrhea followed by diffuse hemorrhages, delirium, shock, jaundice, thrombocytopenia, lymphocytopenia, neutrophilia, focal necrosis in various organs (e.g., kidneys and liver), and acute respiratory distress, among other symptoms, syndromes, and complications described herein.

Aspects of the disclosure are now described with additional detail and options as follows: (i) SARS-CoV-2 Vaccine Antigens; (ii) cTRP Scaffolds; (iii) Recombinant Production; (iv) Compositions for Administration; (v) Methods of Use; (vi) Kits; (vii) Exemplary Embodiments; and (viii) Closing Paragraphs. These headings are provided for organizational purposes only and do not limit the scope or interpretation of the disclosure.

(i) SARS-CoV-2 Vaccine Antigens. SARS-CoV-2 vaccines disclosed herein can include a SARS-CoV-2 antigen. SARS-CoV-2 has been sequenced and has been assigned GenBank accession number MN908947.3. It consists of a single-stranded RNA sequence that is 29,903 base pairs. To date, 10 open reading frames (ORFs) have been identified, including genes encoding a spike (S) protein, an envelope (E) protein, and an (M) protein.

The S protein contains three segments: a large ectodomain, a single-pass transmembrane anchor, and a short intracellular tail. The ectodomain consists of a receptor binding subunit SI and a membrane-fusion subunit S2. Electron microscopy studies revealed that the spike is a clove-shaped trimer with three SI heads and a trimeric S2 stalk. During virus entry, SI binds to a receptor on the host cell surface for viral attachment, and S2 fuses the host and viral membranes, allowing viral genomes to enter host cells. Such receptor binding and membrane fusion are the initial and critical steps in the coronavirus infection cycle.

The amino acid sequence of the SARS-CoV2 spike (S) protein is 1273 amino acids in length. The sequence of the S protein has been reported at GenBank Accession No. QHD43416. Reference to residue positioning in this disclosure is in relation to this reference sequence. The S protein coding sequence is located at nucleic acids 21563-25384 of the SARS-CoV2 genome.

In particular embodiments, the S protein vaccine antigen includes the full length S protein or a fragment thereof. In particular embodiments, the S protein vaccine antigen includes one or more amino acid substitutions or deletions as compared to the sequence reported at GenBank Accession No. QHD43416. Exemplary mutations include
K417T, E484K or N501Y;
K417T, E484K, and N501Y;
K417N;
D614G, A570D, P681H, T716I, S982A, D1118H, K417N or K417T:
D215G, A701V, L18F, R2461, Y453F, 1692V, M1229I, N439K, A222V, S477N, or A376T;
a deletion of one or more residues H69, V70, or Y144;
deletion of residues 242-244;
N501Y, A570D, D614G, P681H, T716I, S982A, and D1118H, deletion of residues 69-70, and deletion of residue Y144;
N501Y, K417N, K417T, E484K, D80A, A701V, and L18F and deletion of residues 242-244;
N439K, T478I, S477N, P479S, V367F, V341I, P330S, P384L, and A522V; or
D614G, D936Y, P1263L, LSF, N439K, R21I, D839Y, L54F, A879S, L18F, F1121L, R847K, T478I, A829T, Q675H, S477N, H49Y, T29I, G769V, G1124V, V1176F, K1073N, P479S, S1252P, Y145, E583D, R214L, A1020V, Q1208H, D215G, H146Y, S98F, T95I, G1219C, A846V, 1197V, R102I, V367F, T572I, A1078S, A831V, P1162L, T73I, A845S, G1219V, H245Y, L8V, Q675R, S254F, V483A, Q677H, D138H, D80Y, M1237T, D1146H, E654D, H655Y, S50L, S939F, S943P, G485R, Q613H, T76I, V341I, M153I, S221L, T859I, W258L, L242F, P681L, V289I, A520S, V1104L, V1228L, L176F, M1237I, T307I, T716I, L14I, M1229I, A1087S, P26S, P330S, P384L, R765L, S940F, T323I, V826L, E1202Q, L1203F, L611F, V615I, A262S, A522V, A688V, A706V, A892S, E554D, Q836H, T1027I, T22I, A222V, A27S, A626V, C1247F, K1191N, M731I, P26L, S1147L, S1252F, S255F, V1264L, V308L, D80A, 1670L, P251L, P631S, *1274Q, A344S, A771S, A879T, D1084Y, D253G, H1101Y, L1200F, Q14H, Q239K, A623V, D215Y, E1150D, G476S, K77M, M177I, P812S, S704L, T51I, T547I, T791I, V1122L, Y145H, D574Y, G142D, G181V, 1834T, N370S, P812L, S12F, T791P, V90F, W152L, A292S, A570V, A647S, A845V, D1163Y, G181R, L84I, L938F, P1143L, P809S, R78M, T1160I, V1133F, V213L, V615F, A831V, D839Y, D839N, D839E, S943P, P1263L, or V622F.

In certain aspects, the S protein vaccine antigen includes one or more proline substitutions that stabilize the S protein turner in the prefusion conformation. For example, proline substitutions can occur at or near the boundary between a Heptad Repeat 1 (HR1) and a central helix of the promoters of the S ectodomain turner. In some embodiments, the proline substitutions occur between residues 970 to 990 (GAISSVLNDILSRLDKVEAE) (SEQ ID NO: 10).

In some embodiments, the S protein vaccine antigen includes two proline substitutions, one at K986 and one at V987.

In certain aspects, the S protein vaccine antigen includes an S1+S2 fragment lacking the carboxy terminus of the S protein (referred to herein as the SI+S2 fragment). In some embodiments, the SI+S2 fragment includes residues 1 to 1213 of the SARS-CoV2 S protein.

In some embodiments, the SI+S2 fragment includes residues 1 to 1213 and two proline substitutions, one at residue K986 and one at residue V987.

In some embodiments, the SI+S2 fragment includes residues 1 to 1213 of the SARS-CoV2 S protein with K417T, E484K, and N501Y substitutions.

In some embodiments, the SI+S2 fragment includes residues 1 to 1213 and includes two proline substitutions, one at residue 986 and one at residue 987, and also includes substitutions K417T, E484K, and N501Y.

In some embodiments, the SI+S2 fragment includes residues 1 to 1213 and one or more substitutions selected from D614G, A570D, P681H, T716I, S982A, D1118H, K417N, K417T, D215G, A701V, L18F, R2461, Y453F, 1692V, M1229I, N439K, A222V, S477N, and A376T.

In some embodiments, the SI+S2 fragment further includes deletion of residues 242-244.

In some embodiments, the SI+S2 fragment includes deletion of residues 69-70 and Y144 and substitutions N501Y, A570D, D614G, P681H, T716I, S982A, and D1118H.

In certain aspects, the S protein vaccine antigen includes the receptor binding domain (RBD, including residues 319-541 of the spike protein). In some embodiments, a fragment of the RBD includes residues 327 to 524 of the S protein, residues 331 to 524 of the S protein, residues 504 to 524 of the S protein or, residues 473 to 490 of the S protein. Particular embodiments include an RBD consensus sequence.

In certain aspects, two or more linear epitopes of the S protein are included, wherein the two or more linear epitopes are separated by a spacer, for example GPGPG (SEQ ID NO: 11). The linear epitopes can include the same or different S protein peptides, for example (RBD Seq. 1-spacer-RBD Seq. 2), wherein RBD Seq. 1 is a first S protein RBD peptide, and RBD Seq. 2 is a second S protein RBD peptide. In some embodiments, the S protein vaccine antigen includes a tandem repeat sequence, for example (RBD-spacer-RBD-spacer)x or (RBD Seq. 1-spacer-RBD Seq. 2-spacer)x, wherein x=2, 3, 4, 5, 6, 7, 8, 9, 10.

Envelope (E) Protein. The E protein is the smallest of the major structural proteins. The amino acid sequence of the SARS-CoV2 envelope (E) protein is 75 amino acids in length and has been reported at GenBank Accession number QHD43418. Its coding sequence has been reported at GenBank Accession No. MN908947.3, and is located at nucleic acids 26245 to 26472 of the SARS-CoV2 genome.

Membrane (M) Protein. The coronavirus M protein is the most abundant structural protein and defines the shape of the viral envelope. The amino acid sequence of the SARS-CoV2 membrane (M) protein is 222 amino acids in length and has been reported at GenBank Accession No. QHD43419. Its coding sequence has been reported at GenBank Accession No. MN908947.3 and is located at nucleic acids 26523 to 27191 of the SARS-CoV2 genome.

Combinations of the SARS-CoV-2 vaccine antigens can also be used.

In certain examples, the vaccine antigen includes the sequence as set forth in SEQ ID NO: 1 or SEQ ID NO: 5 or has a sequence with at least 90% sequence identity to the sequence as set forth in SEQ ID NO: 1 or SEQ ID NO: 5.

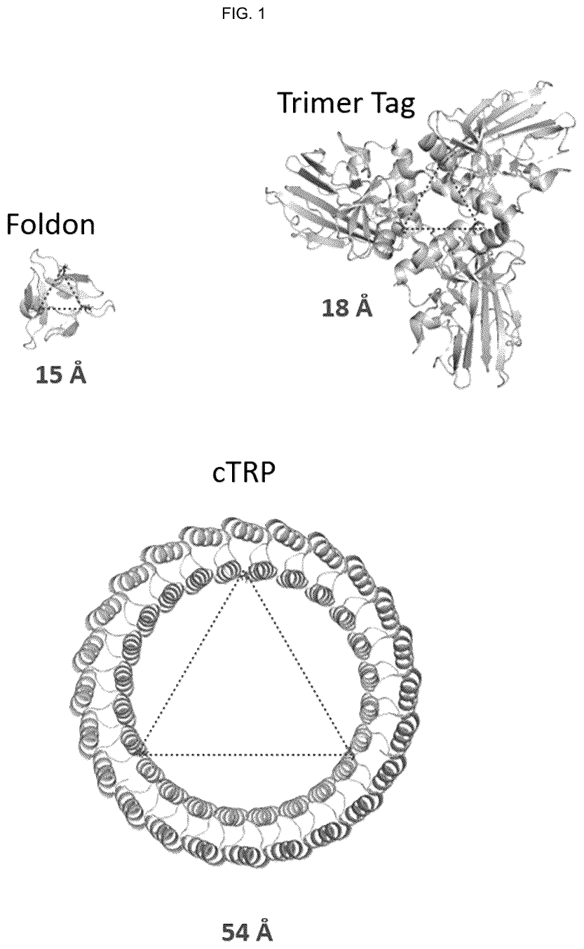

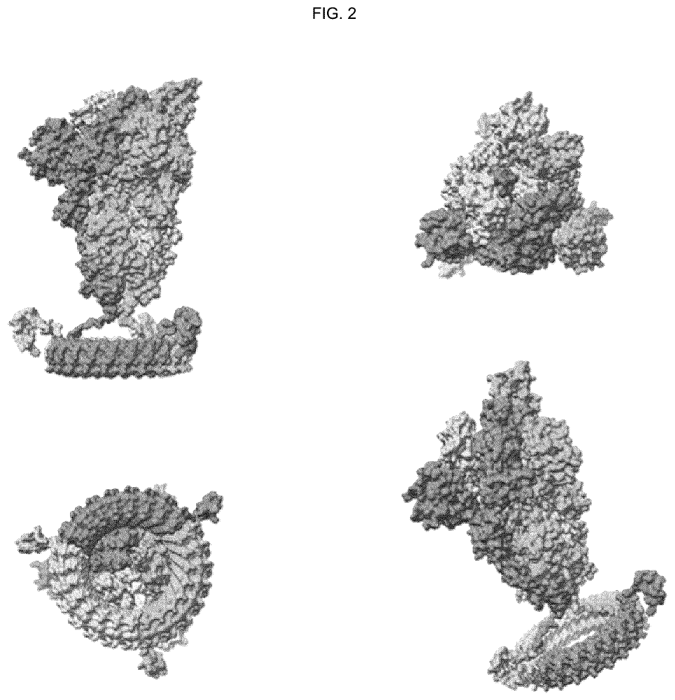

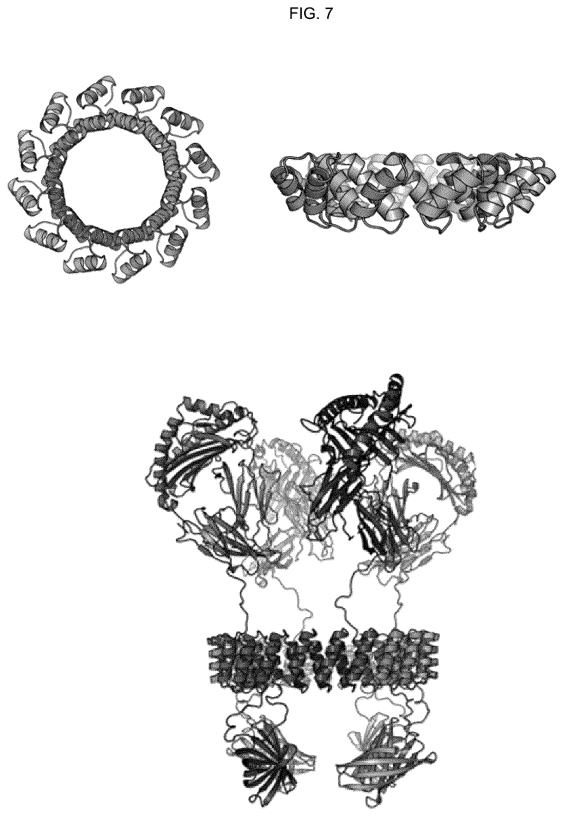

(ii) cTRP Scaffolds. As indicated, SARS-CoV-2 vaccine antigens are presented on a cTRP scaffold. cTRPs include repetitive α-helical structures joined by linkers. Referring to, for example, FIGS. 1 and 7, each repetitive α-helical structure includes an outer α-helix and an inner α-helix. In particular embodiments, an α-helical structure is repetitive (e.g., structurally repetitive) when following (i) stacking with an adjacent α-helical structure; and (ii) comparison using root-mean-square-deviation (RMSD), the distance between corresponding atoms of the stacked outer α-helices and the stacked inner α-helices is within 2 angstrom (A); within 1.5 Å; within 1 Å; within 0.5 Å; within 0.4 Å; or within 0.2 Å.

Exemplary amino acid sequences that generate an α-helix include VEELLKLAKAAYYS (SEQ ID NO: 52); VEEAYKLALKL (SEQ ID NO: 53); VEELLKLAEAAYYS (SEQ ID NO: 54); PTEALLKLIAEAK (SEQ ID NO: 206); ETEAKEEAEKALKE (SEQ ID NO: 207); STEAKEEAIKALKE (SEQ ID NO: 208); ELEAKVLAEKALKE (SEQ ID NO: 209); ETEAKLEAEKALKE (SEQ ID NO: 210); PTEVLLELIAEAS (SEQ ID NO: 17); KEEVKEKFLKELSK (SEQ ID NO: 211); KEEVKRKFLKELSK (SEQ ID NO: 212); KAEVKREFLWELSL (SEQ ID NO: 213); KEEVKEKFLAELEK (SEQ ID NO: 214); REEVKEKFLKELRK (SEQ ID NO: 18); KEEVKEKFLKELSF (SEQ ID NO: 19); KEEVKKKFWKELSL (SEQ ID NO: 20); KREVKRWFLFELRK (SEQ ID NO: 215); KAEVKLKFLFELSF (SEQ ID NO: 216); KEEVKEKFLKELFK (SEQ ID NO: 217); TTEALLILIAEAS (SEQ ID NO: 218); VEQQKQRFKELVKK (SEQ ID NO: 219); TAIAQILAIKASAK (SEQ ID NO: 25); TELERALRYAKKV (SEQ ID NO: 220); TELERALRYAVKV (SEQ ID NO: 26); TELEQALRYAKFV (SEQ ID NO: 27); LELTRALAYAKKV (SEQ ID NO: 28); TELERALRYAKLV (SEQ ID NO: 29); TELERALRYAKYV (SEQ ID NO: 30); PELEYALAYAKKV (SEQ ID NO: 31); TELERALIFAEAV (SEQ ID NO: 221); TELDRALWYAKKV (SEQ ID NO: 222); TELERALLYAKKV (SEQ ID NO: 223); TELERALAYARLV (SEQ ID NO: 224); TELERALRYAEKV (SEQ ID NO: 225); TELERALWYAKKV (SEQ ID NO: 226); SAIATAYIALAEYL (SEQ ID NO: 227); EALLKAIEIAIKL (SEQ ID NO: 228); SAIAEAYIALARYL (SEQ ID NO: 229); SALAQILAIYASAY (SEQ ID NO: 230); TLFLRALKLAKEV (SEQ ID NO: 231); ELYIRVLAIVAEAE (SEQ ID NO: 232); TKLELALKLALKK (SEQ ID NO: 233); KLYIEVLAIVAEAE (SEQ ID NO: 234); ELYIRVLAIVAKAE (SEQ ID NO: 235); KLYIEVLAIVAKAE (SEQ ID NO: 236); LEQALKILKVAAEL (SEQ ID NO: 39); VEEAVKRALKLKTKL (SEQ ID NO: 40); LEQALKILEVAAEL (SEQ ID NO: 41); LEQALKILEVAAKL (SEQ ID NO: 42); VEEAVKRAMKLKTKL (SEQ ID NO: 43); as well as SEQ ID NO: 58-63; 73, 74, 80 and 81.

Each repetitive α-helical structure includes 2 sequences that each form an α helix. The two sequences forming a helices within each repetitive structure can be identical or can have at least 99%, at least 98%, at least 97%, at least 96%, at least 95%, at least 94%, at least 93%, at least 92%, at least 91%, at least 90% or at least 85% sequence identity to the other within the structure. Thus, in particular embodiments, a repetitive α-helical structure of a cTRP disclosed herein would include at least two sequences from SEQ ID NOs. 17-20, 25-31, 39-43, 52-54, 206-236, 58-63, 73, 74, 80 and 81 to generate an outer α helix and an inner α helix, respectively.

Many cTRPs have a circular architecture stabilized by interactions between the first and last repeats, obviating the need for capping repeats to maintain solubility and making them more tolerant than open repeat architectures to imperfections in the designed geometry. Thus, in particular embodiments, a circular protein (e.g., a protein having a circular architecture) is one wherein the N-terminal and C-terminal ends of the protein are naturally found within 10 Å following expression and folding. In particular embodiments, a circular protein is one wherein the N-terminal and C-terminal ends of the protein are naturally found within 10 Å; within 9 Å; within 8 Å; within 7 Å; within 6 Å; within 5 Å; within 4 Å; within 3 Å; within 2 Å; within 1 Å; or within 0.5 Å; following expression and folding. Naturally found means that the cTRP is self-folding. In particular embodiments, a circular protein is one designated as such by the teachings of Kajava, A. V.

Tandem repeats in proteins: from sequence to structure. J. Struct. Biol. 179, 279-288, doi:10.1016/j.jsb.2011.08.009 (2012).

Many cTRPs are left-handed proteins. In particular embodiments, to compute the handedness of helical bundles formed by cTRPs, an approximate helical bundle axis curve can be generated by joining the location of repeat-unit centers of mass in a sliding fashion along the protein chain. The handedness can then be determined by computing the directionality of the winding of the polypeptide chain about this axis curve. In particular embodiments, a left-handed protein is one wherein the protein is designated as such by the teachings of Kajava, A. V. Tandem repeats in proteins: from sequence to structure. J. Struct. Biol. 179, 279-288, doi:10.1016/j.jsb.2011.08.009 (2012).

In particular embodiments, the left-handedness of particular cTRPs is due in part to the use of inter-helical turns whose geometry naturally imparts a handedness to the resulting helical bundle. The 3-residue ‘GBB’ (αL-β-β) turn type used in particular embodiments prefers a left-handed dihedral twist between the connected helices, while a ‘GB’ turn can result in a right-handed geometry. Both these turn types are also compatible with canonical helix capping interactions.

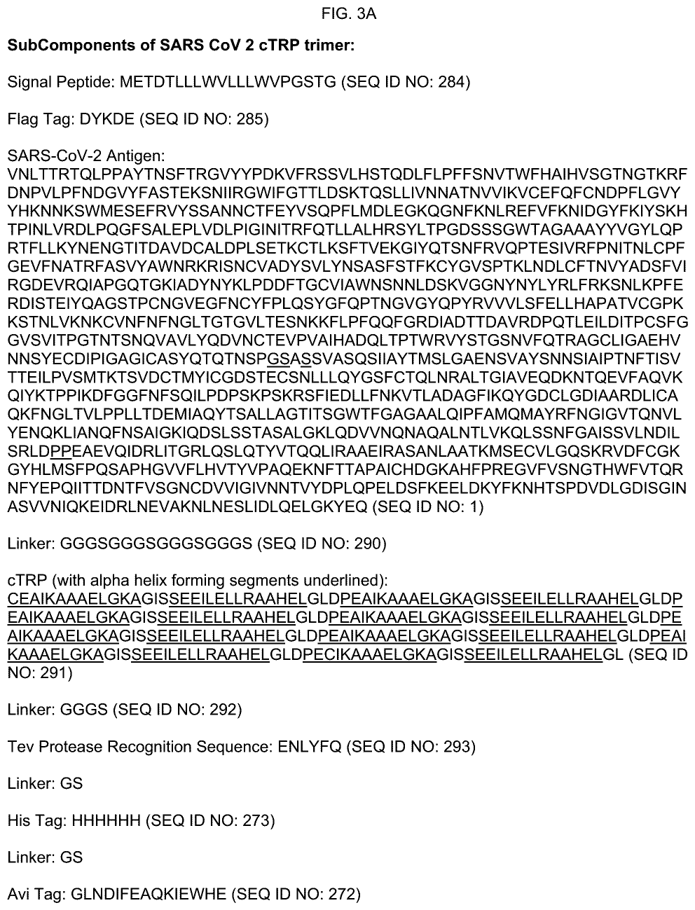

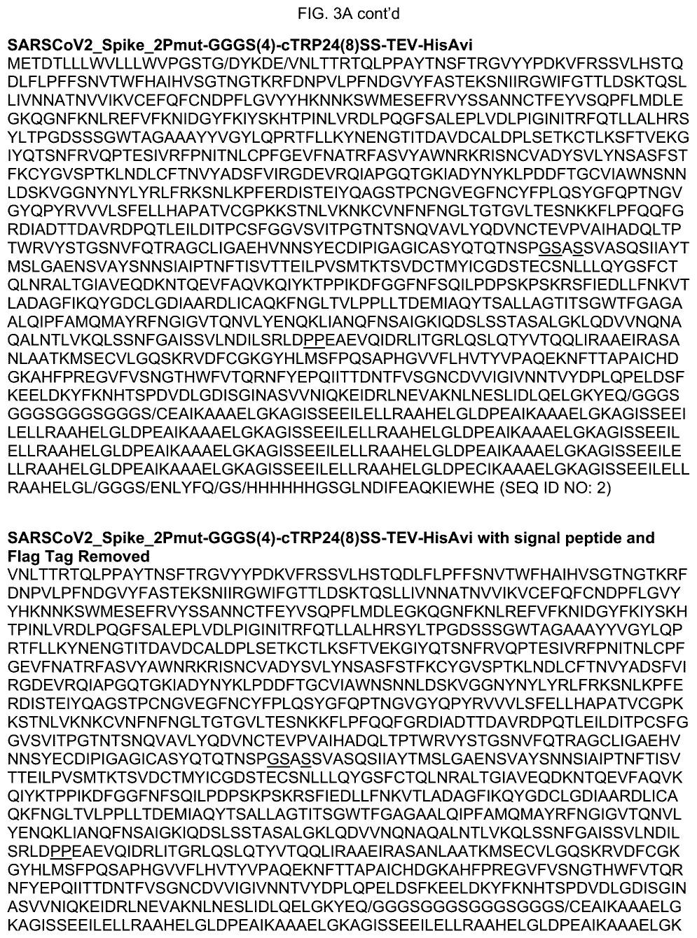

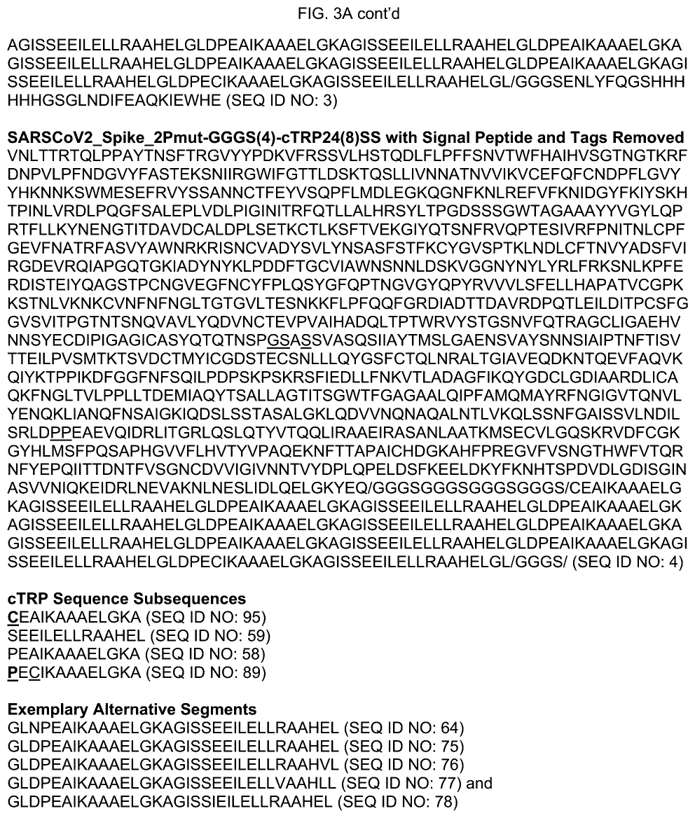

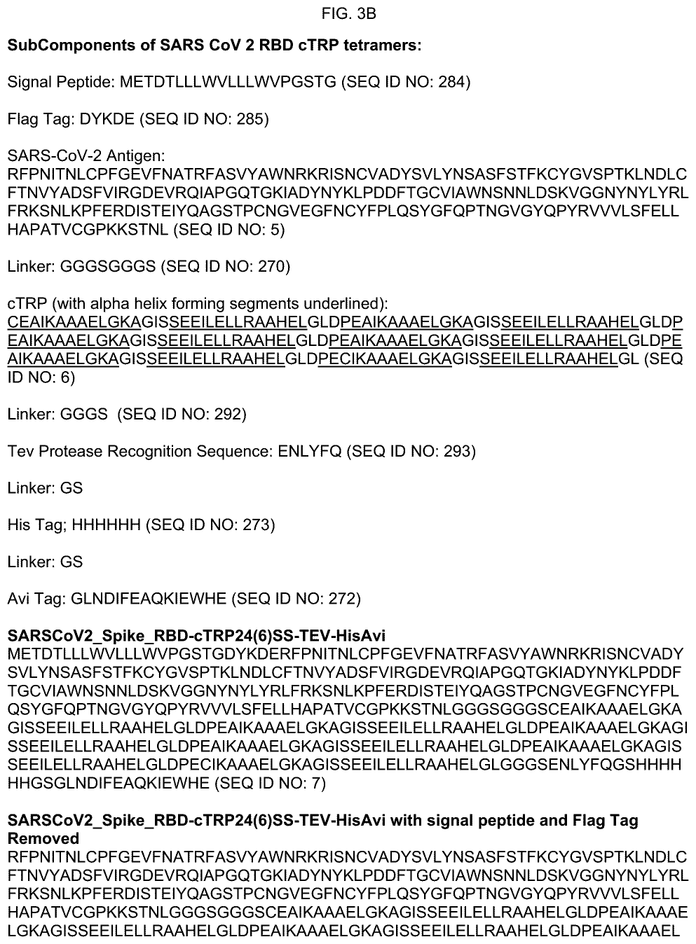

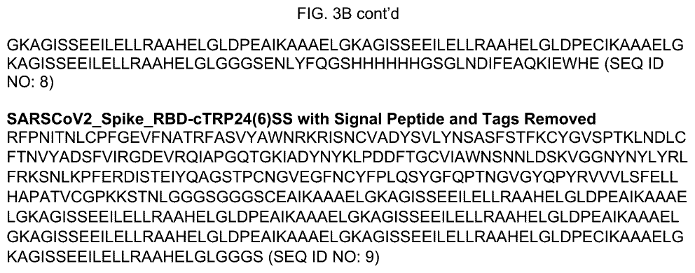

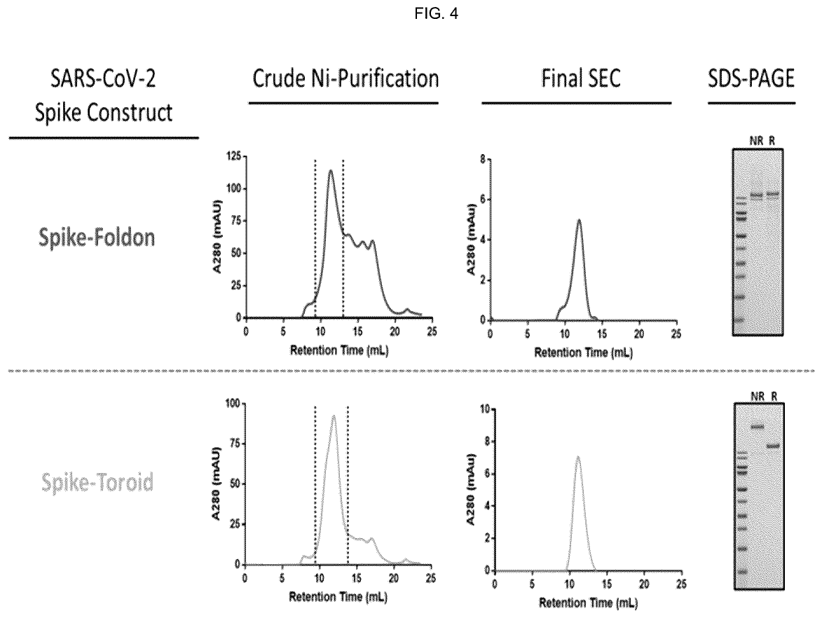

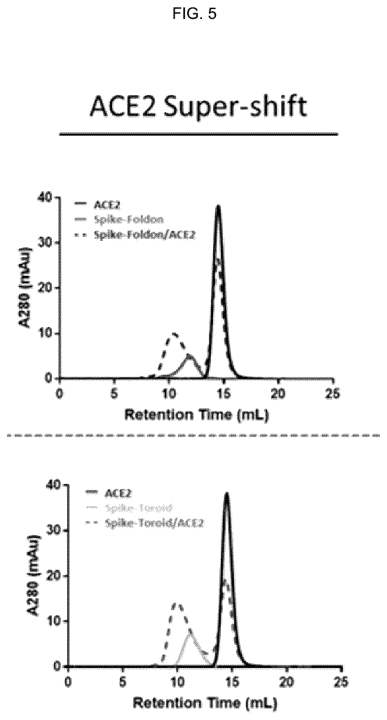

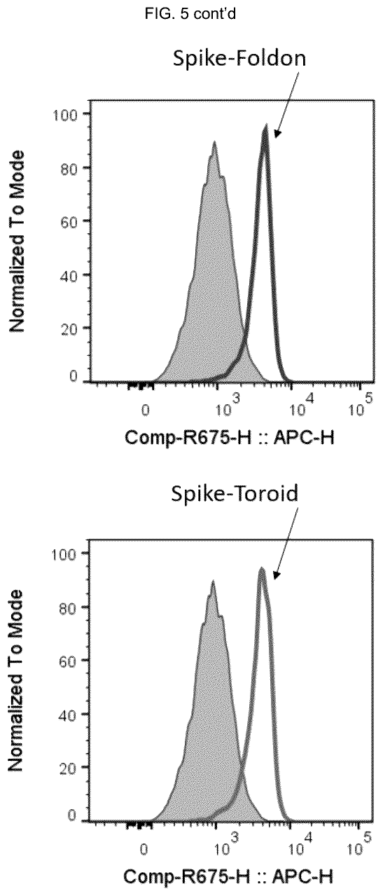

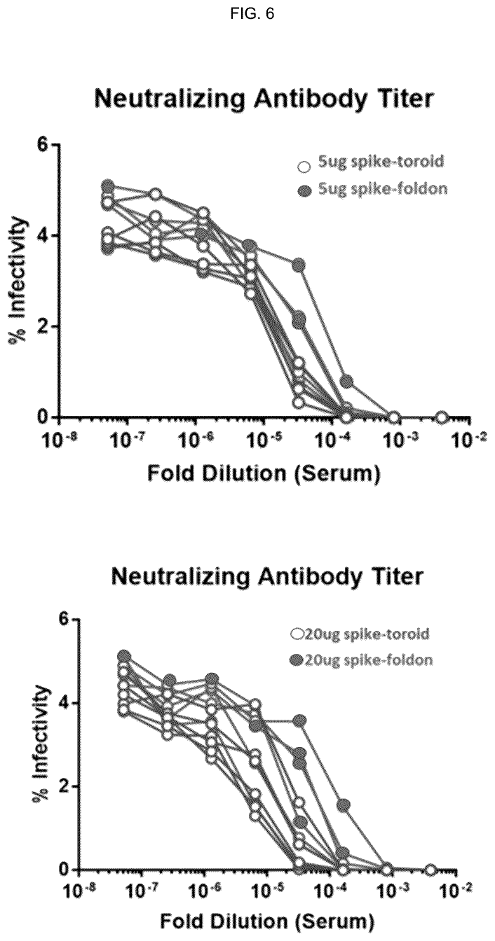

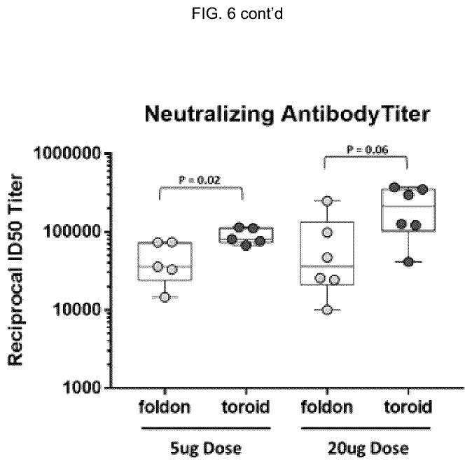

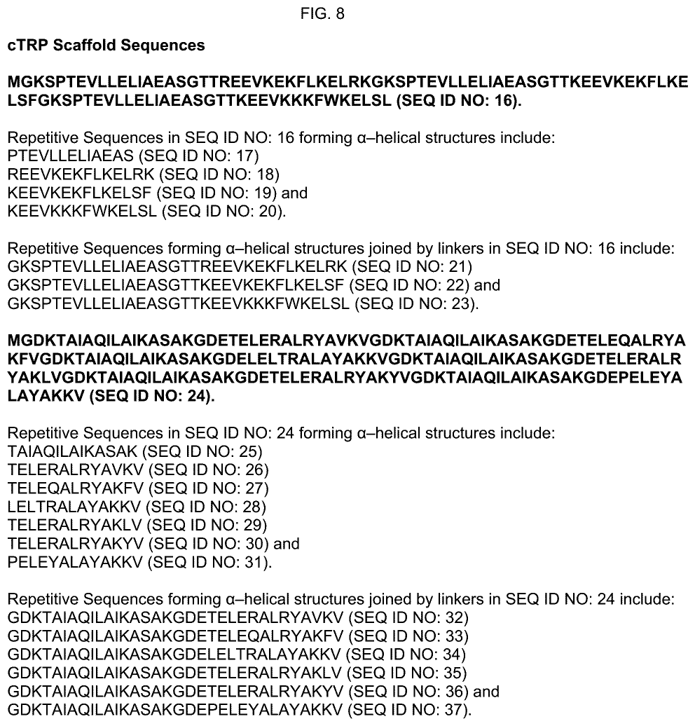

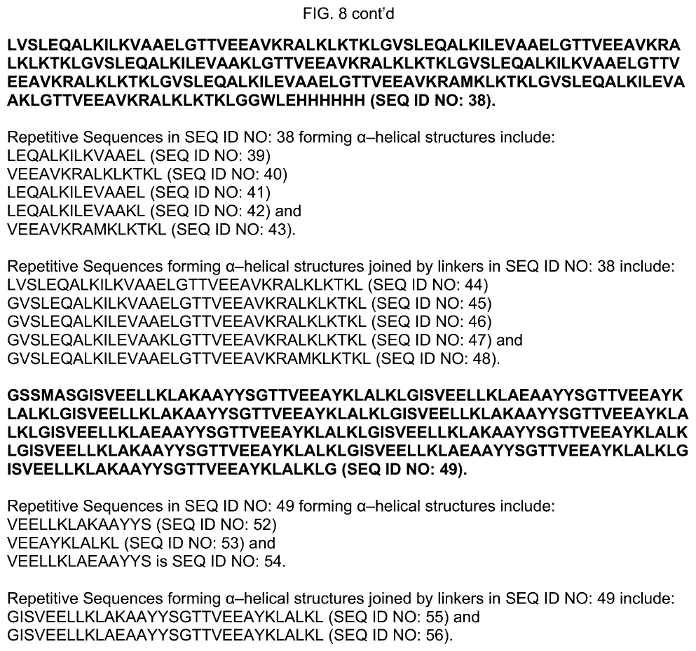

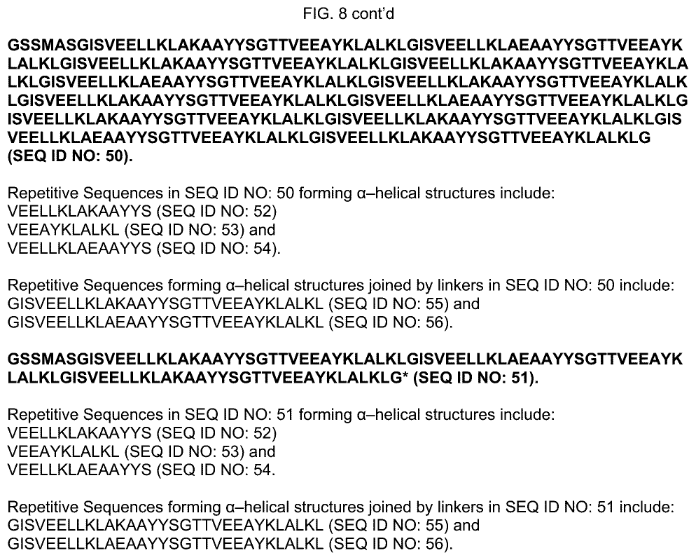

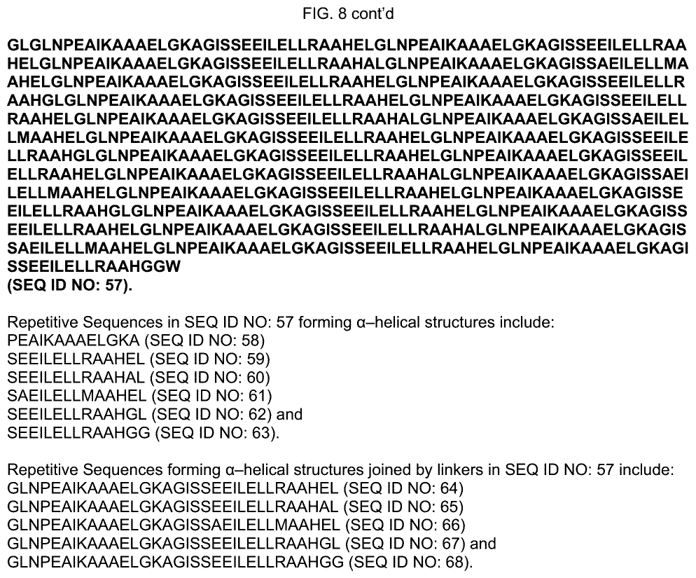

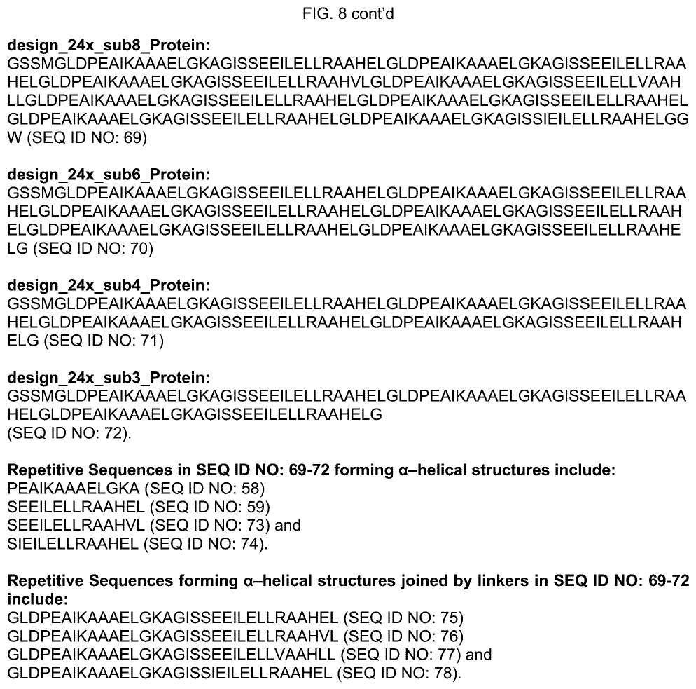

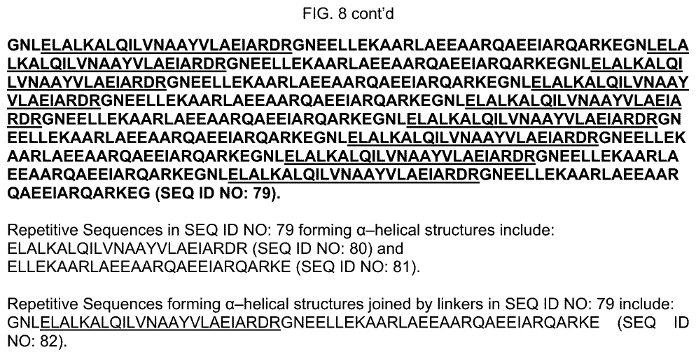

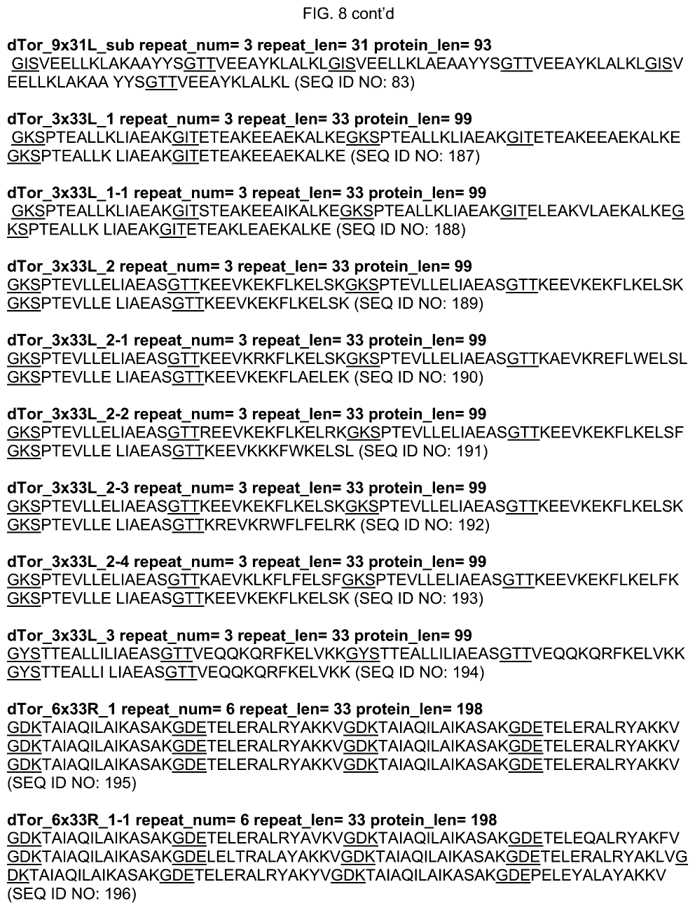

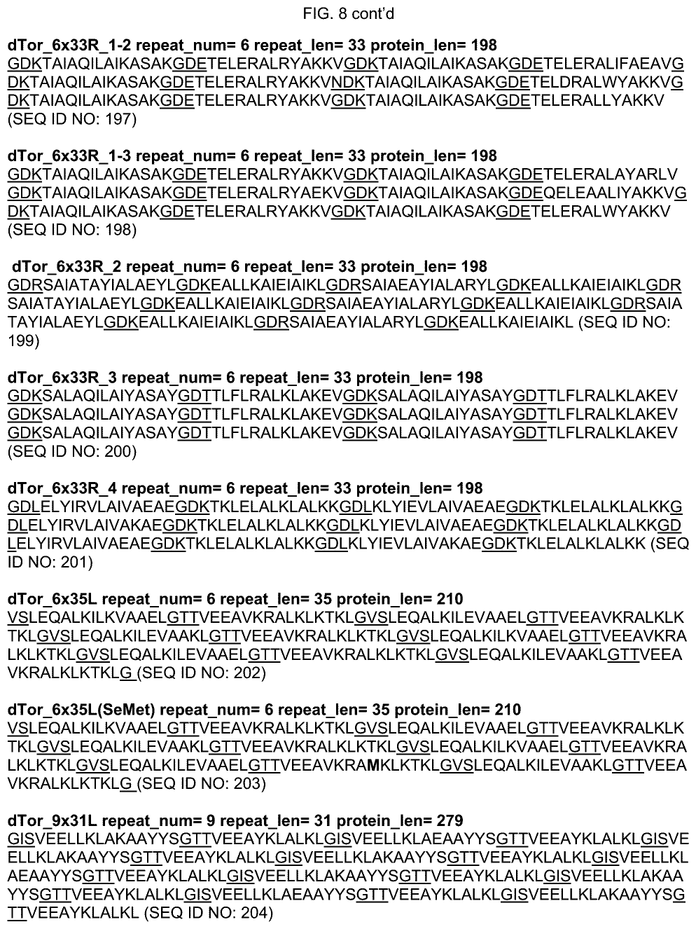

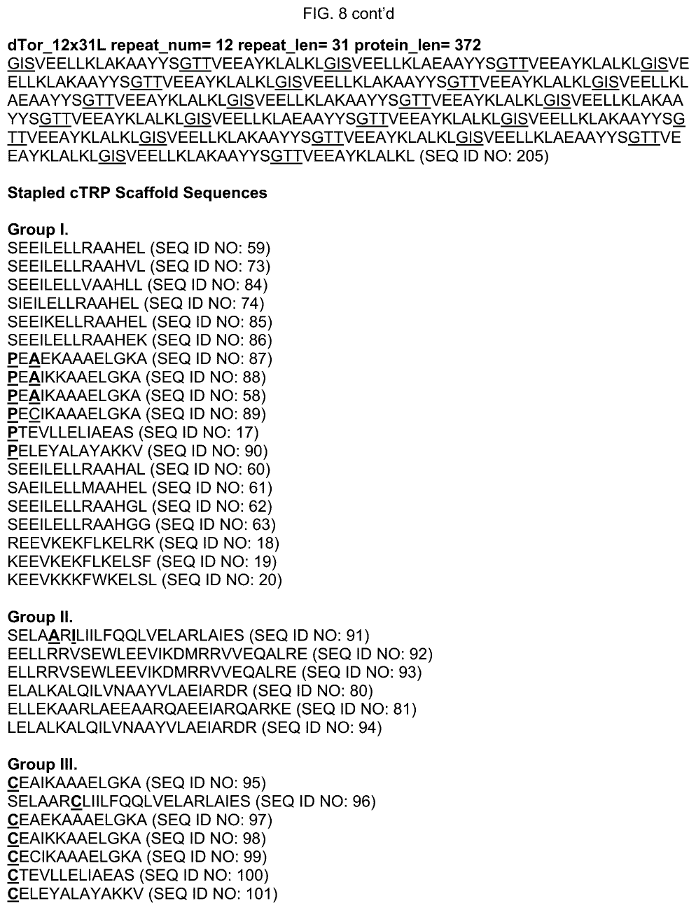

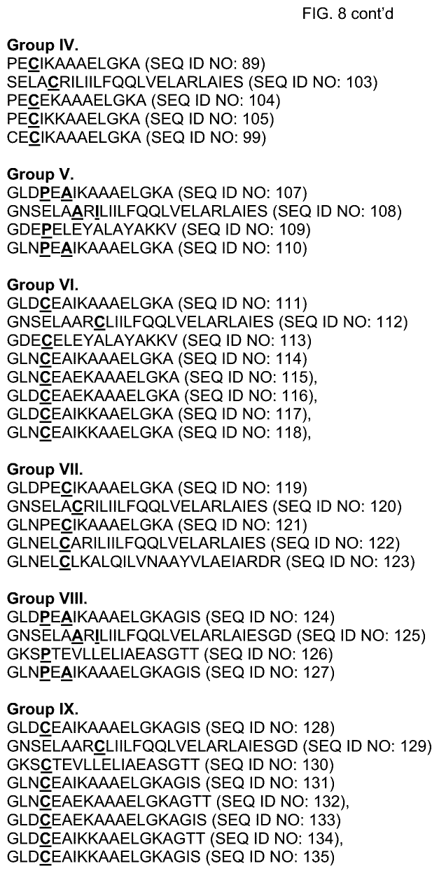

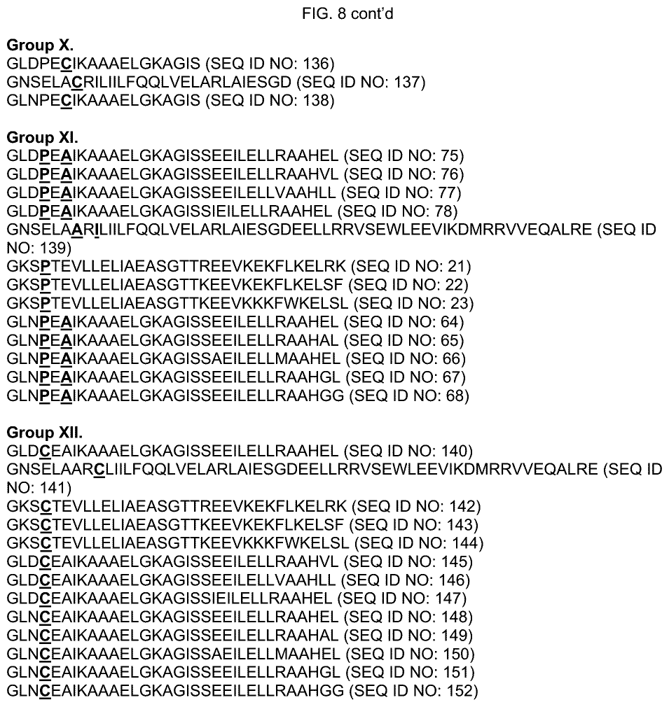

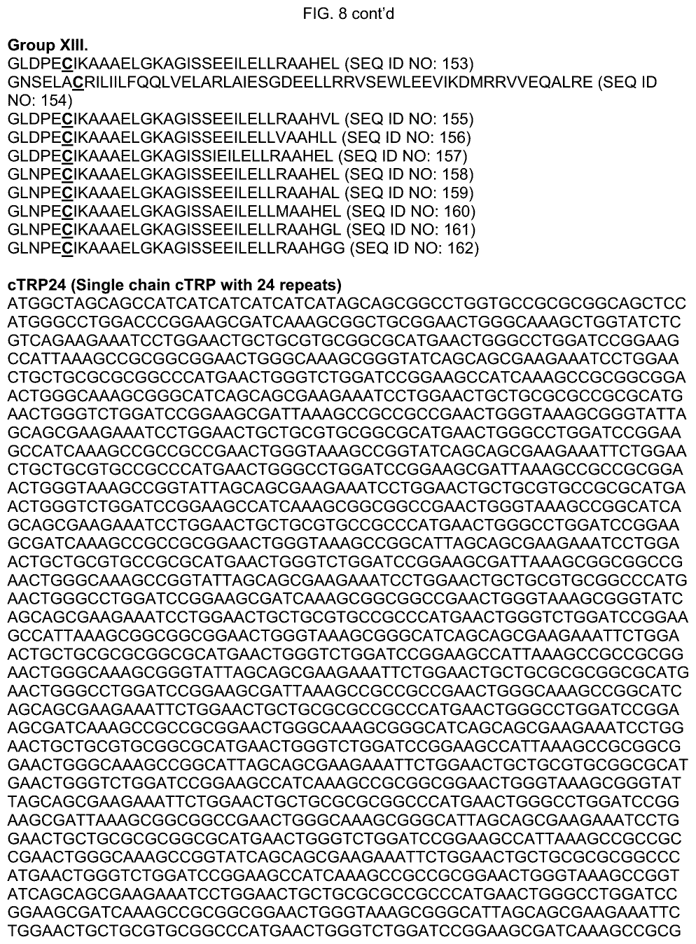

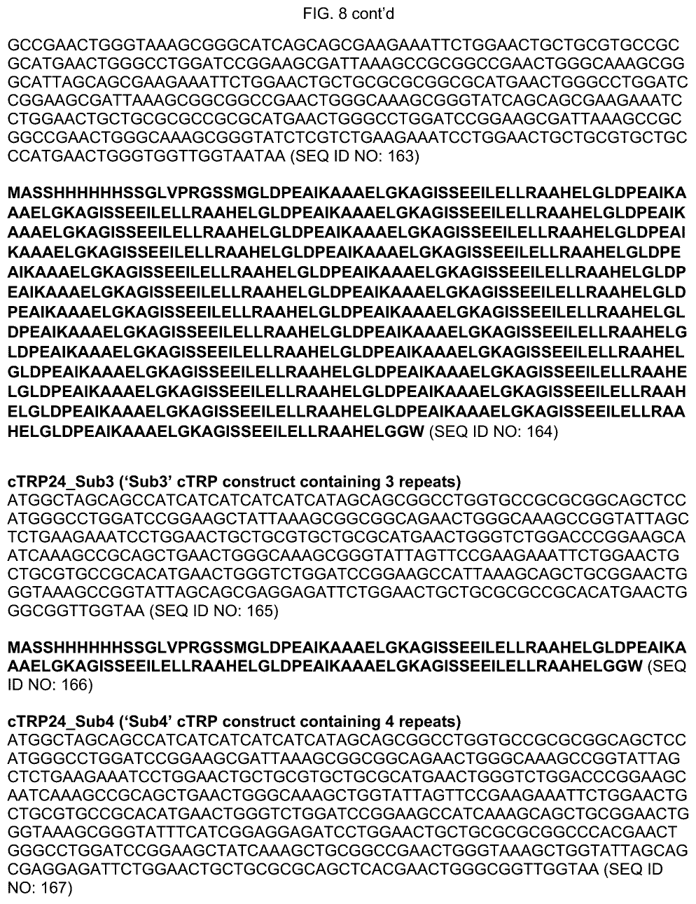

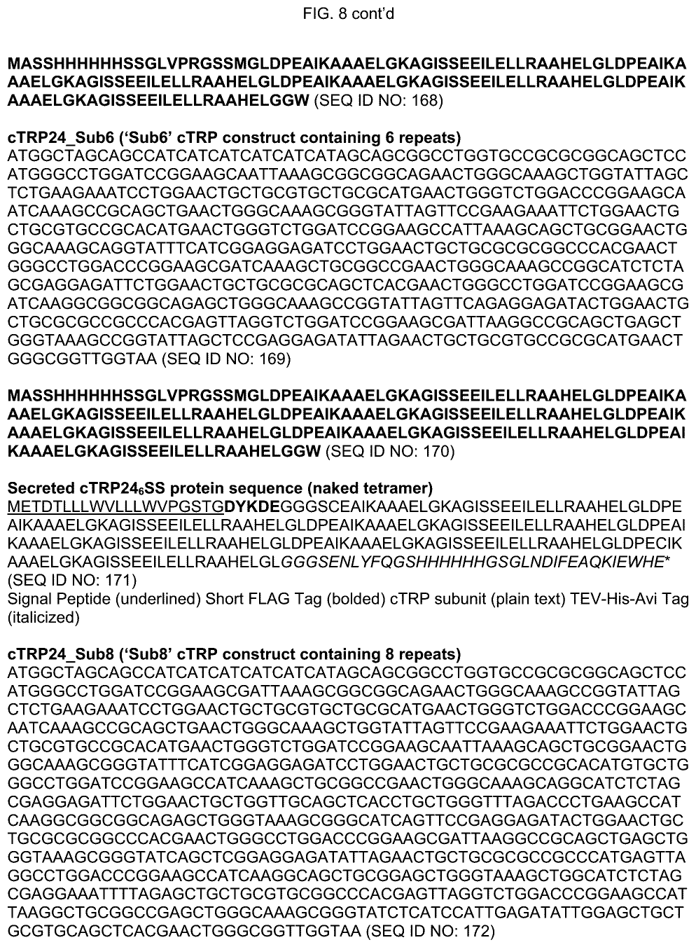

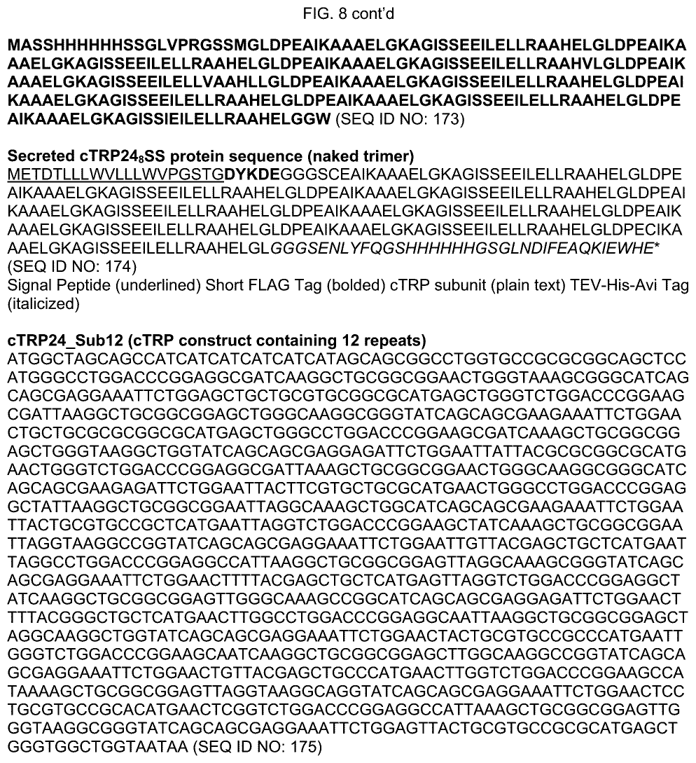

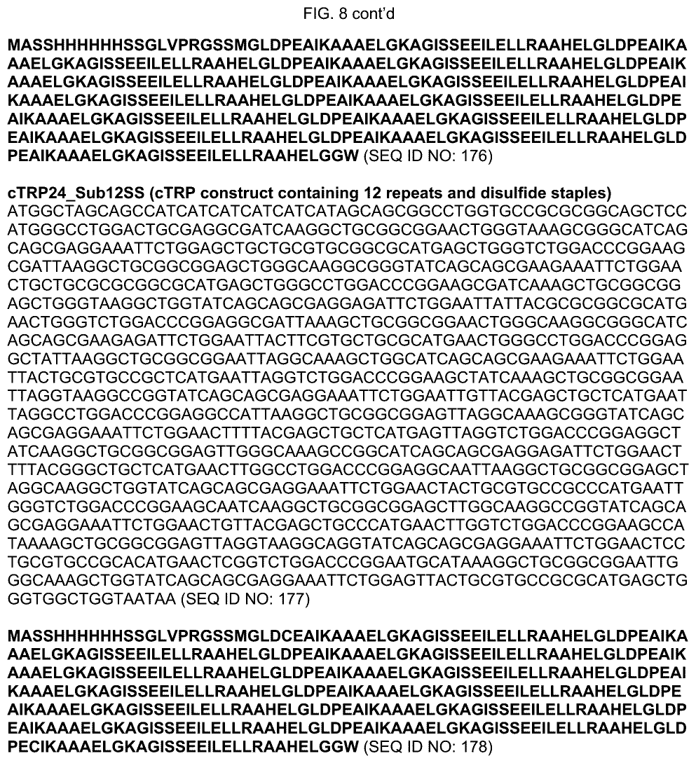

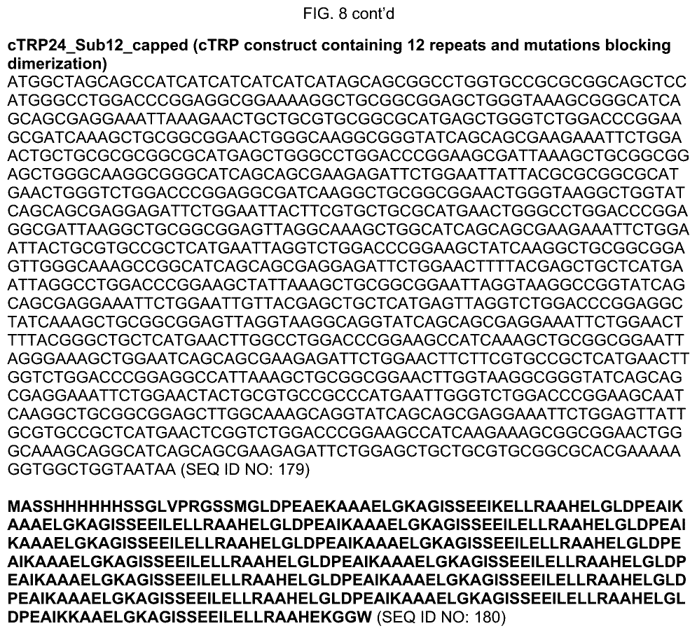

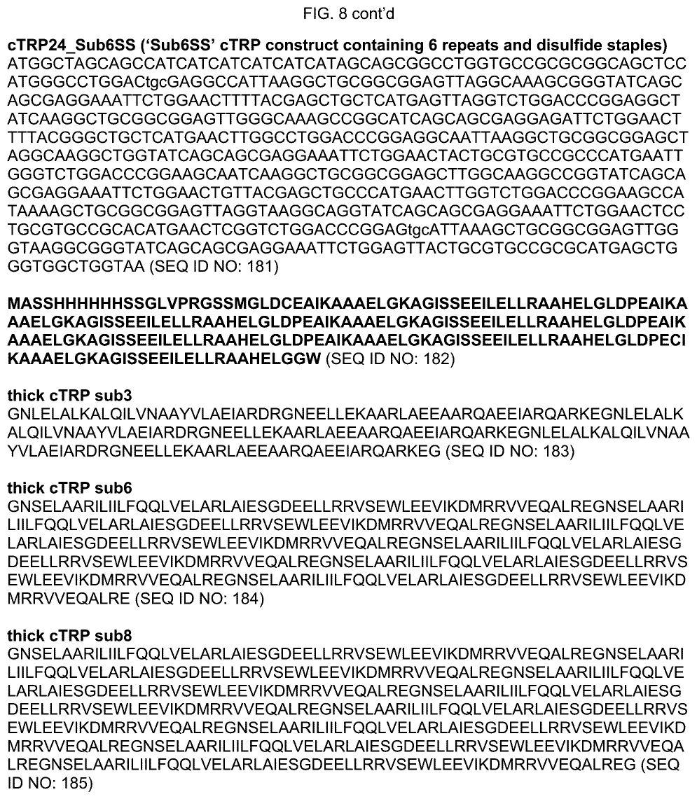

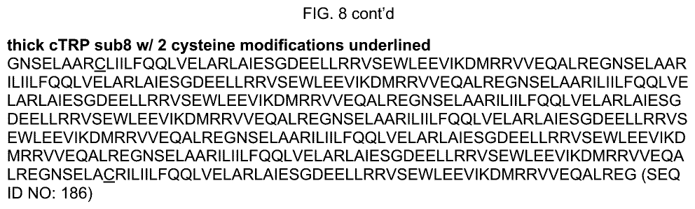

Based on the foregoing, and as stated, in particular embodiments linkers between α helix residues can utilize a GBB format. In particular embodiments, the G residue is glycine. In particular embodiments, the G residue is not isoleucine or valine. In particular embodiments, the B residues are selected from serine, threonine, asparagine, or glutamine. Examples of GBB linkers include GKS; GIT; GTT; GYS; GDK; GDE; NDK; GDR; GDL; and GIS (see, e.g., FIGS. 3A, 3B, and 8). As will be understood by one of ordinary skill in the art, particular residues that fall within a G or B classification can depend on the particular protein at issue. Therefore, while representative (and common) selection options within these groups are provided, such examples are not exclusive to use of other potential residues. Without being bound by theory, and in particular embodiments, GBB linkers are utilized because they facilitate formation of left-handed proteins. Referring to FIGS. 3A, 3B and 8, it is important to note that some cTRP scaffolds are presented as starting with an alpha-helical forming sequence, while others begin with a linker. Due to the circular architecture of cTRPs, these repeat proteins can “begin” or “end” with either segment type at the N- or C-terminus.

In particular embodiments, repetitive α-helical structures joined by linkers can be formed from sequences selected from:

(SEQ ID NO: 55)
GISVEELLKLAKAAYYSGTTVEEAYKLALKL;
(SEQ ID NO: 56)
GISVEELLKLAEAAYYSGTTVEEAYKLALKL;
(SEQ ID NO: 239)
GKSPTEALLKLIAEAKGITETEAKEEAEKALKE;
(SEQ ID NO: 240)
GKSPTEALLKLIAEAKGITSTEAKEEAIKALKE;
(SEQ ID NO: 241)
GKSPTEALLKLIAEAKGITELEAKVLAEKALKE;
(SEQ ID NO: 242)
GKSPTEALLKLIAEAKGITETEAKLEAEKALKE;
(SEQ ID NO: 243)
GKSPTEVLLELIAEASGTTKEEVKEKFLKELSK;
(SEQ ID NO: 244)
GKSPTEVLLELIAEASGTTKEEVKRKFLKELSK;
(SEQ ID NO: 245)
GKSPTEVLLELIAEASGTTKAEVKREFLWELSL;
(SEQ ID NO: 246)
GKSPTEVLLELIAEASGTTKEEVKEKFLAELEK;
(SEQ ID NO: 21)
GKSPTEVLLELIAEASGTTREEVKEKFLKELRK;
(SEQ ID NO: 22)
GKSPTEVLLELIAEASGTTKEEVKEKFLKELSF;
(SEQ ID NO: 23)
GKSPTEVLLELIAEASGTTKEEVKKKFWKELSL;
(SEQ ID NO: 247)
GKSPTEVLLELIAEASGTTKREVKRWFLFELRK;
(SEQ ID NO: 248)
GKSPTEVLLELIAEASGTTKAEVKLKFLFELSF;
(SEQ ID NO: 249)
GKSPTEVLLELIAEASGTTKEEVKEKFLKELFK;
(SEQ ID NO: 250)
GYSTTEALLILIAEASGTTVEQQKQRFKELVKK;
(SEQ ID NO: 251)
GDKTAIAQILAIKASAKGDETELERALRYAKKV;
(SEQ ID NO: 32)
GDKTAIAQILAIKASAKGDETELERALRYAVKV;
(SEQ ID NO: 33)
GDKTAIAQILAIKASAKGDETELEQALRYAKFV;
(SEQ ID NO: 34)
GDKTAIAQILAIKASAKGDELELTRALAYAKKV;
(SEQ ID NO: 35)
GDKTAIAQILAIKASAKGDETELERALRYAKLV;
(SEQ ID NO: 36)
GDKTAIAQILAIKASAKGDETELERALRYAKYV;
(SEQ ID NO: 37)
GDKTAIAQILAIKASAKGDEPELEYALAYAKKV;
(SEQ ID NO: 252)
GDKTAIAQILAIKASAKGDETELERALIFAEAV;
(SEQ ID NO: 253)
NDKTAIAQILAIKASAKGDETELDRALWYAKKV;
(SEQ ID NO: 254)
GDKTAIAQILAIKASAKGDETELERALLYAKKV;
(SEQ ID NO: 255)
GDKTAIAQILAIKASAKGDETELERALAYARLV;
(SEQ ID NO: 256)
GDKTAIAQILAIKASAKGDETELERALRYAEKV;
(SEQ ID NO: 257)
GDKTAIAQILAIKASAKGDEQELEAALIYAKKV;
(SEQ ID NO: 258)
GDKTAIAQILAIKASAKGDETELERALWYAKKV;
(SEQ ID NO: 259)
GDRSAIATAYIALAEYLGDKEALLKAIEIAIKL;
(SEQ ID NO: 260)
GDRSAIAEAYIALARYLGDKEALLKAIEIAIKL;
(SEQ ID NO: 261)
GDKSALAQILAIYASAYGDTTLFLRALKLAKEV;
(SEQ ID NO: 262)
GDLELYIRVLAIVAEAEGDKTKLELALKLALKK;
(SEQ ID NO: 263)
GDLKLYIEVLAIVAEAEGDKTKLELALKLALKK;
(SEQ ID NO: 264)
GDLELYIRVLAIVAKAEGDKTKLELALKLALKK;
(SEQ ID NO: 265)
GDLKLYIEVLAIVAKAEGDKTKLELALKLALKK;
(SEQ ID NO: 45)
GVSLEQALKILKVAAELGTTVEEAVKRALKLKTKL;
(SEQ ID NO: 46)
GVSLEQALKILEVAAELGTTVEEAVKRALKLKTKL;
(SEQ ID NO: 47)
GVSLEQALKILEVAAKLGTTVEEAVKRALKLKTKL;
(SEQ ID NO: 48)
GVSLEQALKILEVAAELGTTVEEAVKRAMKLKTKL;
and
(SEQ ID NO: 44)
LVSLEQALKILKVAAELGTTVEEAVKRALKLKTKL.

Additional examples include SEQ ID NO: 64-68; 75-78; and 82.

SEQ ID NOs: 83,187-205; 16, 24, 38, 49-51, 57, 69-72, and 79 provide exemplary repetitively patterned amino acid sequences that create cTRPs. In particular embodiments, adjacent structural repeats can include sequences that are identical or that have at least 99%, at least 98%, at least 97%, at least 96%, at least 95%, at least 94%, at least 93%, at least 92%, at least 91%, at least 90% or at least 85% sequence identity to the adjacent structural repeat. Methods to determine sequence identity are described below.

As will be understood by one of ordinary skill in the art, variants of the cTRP sequences, that do not alter the circular, handed and repetitive structural nature of the proteins can also be used. Indeed, variants of all protein sequences disclosed herein can be used, so long as the variation does not render the protein unfit for its intended purpose.

In addition to being circular, handed, and structurally repetitive, cTRPs exhibit self-folding, high thermostability, and high solubility. Self-folding means that the cTRPs fold without any need for inclusion of additional folding domains or subunits (e.g., additional protein domains physically appended to the cTRP construct, or independently added protein folding chaperones such as GroEL/GroES or redox-dependent folding cofactors such as thioredoxin). High thermostability means that the proteins retain their overall secondary structure (including alpha-helices) and tertiary structures (defined by their size and shape) at temperatures as high as 95° C. High solubility means that the proteins can be concentrated to levels of 1 mg/mL or higher at physiological pH and salt concentrations without formation of soluble protein aggregates or protein precipitate.

cTRPs can be described as having the formula: (a-b-x-y)ₙ wherein a and x represent linker sequences (e.g., GBB linker sequences), b represents an amino acid sequence that forms an alpha (α) helix, y represents an amino acid sequence that forms a second α helix, n=1 or more, each (a-b-x-y) unit is structurally repetitive to an adjacent (a-b-x-y) unit; the protein is handed (e.g., left-handed); and the N- and C-termini of the protein create a circular architecture.

In certain examples, stability of cTRPs can be increased by adopting one of two approaches. The first approach includes selecting amino acid sequences that form an alpha (a) helix and introducing precisely placed cysteine mutations within the N-terminal and C-terminal segments of the b position of the (a-b-x-y)ₙ formula. These precisely placed cysteine mutations create di-sulfide bonds which serve as a “staple” to fully close the proteins circular architecture. That is, the closed self-assembled cTRPs have an N-terminus and a C-terminus that are physically linked rather than simply constrained by inter-repeat packing geometry.

In particular embodiments, a selected α-helix-forming protein has 13 amino acid residues with a cysteine at position 1 for use in the N-terminal b segment of (a-b-x-y)ₙ. In particular embodiments, a selected α-helix-forming protein has 13 amino acid residues with a cysteine at position 3 for use in the C-terminal b segment of (a-b-x-y)ₙ. In particular embodiments, a selected α-helix-forming protein has 13 amino acid residues with a proline at position 1. The position 1 proline can be modified to cysteine in the N-terminal b segment of (a-b-x-y)ₙ. In particular embodiments, a selected α-helix-forming protein has 13 amino acid residues with an alanine at position 3. The position 3 alanine can be modified to cysteine in the C-terminal b segment of (a-b-x-Y)ₙ. These selections and mutations create self-assembled, dosed (“stapled”) cTRPs with increased stability over a non-closed circular architecture.

In particular embodiments, a selected α-helix-forming protein has 25 amino acid residues with a cysteine at position 7 for use in the N-terminal b segment of (a-b-x-y)ₙ. In particular embodiments, a selected α-helix-forming protein has 25 amino acid residues with a cysteine at position 5 for use in the C-terminal b segment of (a-b-x-y)ₙ. In particular embodiments, a selected α-helix-forming protein has 25 amino acid residues with an alanine at position 5. The position 5 alanine can be modified to cysteine in the C-terminal b segment of (a-b-x-y)ₙ. In particular embodiments, a selected α-helix-forming protein has 25 amino acid residues with an isoleucine at position 7. The position 7 isoleucine can be modified to cysteine in the N-terminal b segment of (a-b-x-y)ₙ. These selections and mutations create self-assembled, closed cTRPs with increased stability over a non-dosed circular architecture (see e.g., FIGS. 3A, 3B, and 8).

The second approach to creating self-assembling cTRPs with increased stability includes selecting amino acid sequences that form an α helix wherein the selected amino acid sequences each have at least 23 amino acid residues (creating “thick” self-assembling cTRPs).

The first and second approaches can be practiced together to create self-assembling cTRPs that are both dosed and thick.

SARS-CoV-2 vaccine antigens are presented by a cTRP scaffold. SARS-CoV-2 vaccine antigens can be inserted as functional domains, for example, according to the formula (d-a-b-x-y)ₙ, (a-d-b-x-y)ₙ, (a-b-d-x-y)ₙ, (a-b-x-d-y)ₙ or (a-b-x-y-d)ₙ wherein b and y represent linkers, a represents an amino acid sequence that forms an alpha (α) helix, x represents an amino acid sequence that forms a second α helix, d represents a SARS-CoV-2 vaccine antigen, and n=1 or more. In certain examples, the d segment SARS-CoV-2 antigen can be inserted between residues of a b and/or a y linker sequence.

In particular embodiments, additional linkers can be inserted around a d segment SARS-CoV-2 vaccine antigen to further tailor presentation of the vaccine antigen. Linkers can be used that fuse domains together and result in stably expressed, functional proteins. Examples of linkers can be found in Chen et al., Adv Drug Deliv Rev. 2013 Oct. 15; 65(10): 1357-1369. Linkers can be flexible, rigid, or semi-rigid, depending on the desired SARS-CoV-2 vaccine antigen presentation. Commonly used flexible linkers include Gly-Ser linkers such as GGSGGGSGGSG (SEQ ID NO: 266), GGSGGGSGSG (SEQ ID NO: 267), GGSGGGSG (SEQ ID NO: 268), GGGGSGGGGS (SEQ ID NO: 269); GGGSGGGS (SEQ ID NO: 270); GGSGGS (SEQ ID NO: 271), GGGSGGGSGGGSGGGS (SEQ ID NO: 290), GGGS (SEQ ID NO: 292), and GS.

In some situations, flexible linkers may be incapable of maintaining a distance or positioning of SARS-CoV-2 vaccine antigens needed for a particular use. In these instances, rigid or semi-rigid linkers may be useful. Examples of rigid or semi-rigid linkers include proline-rich linkers. In particular embodiments, a proline-rich linker is a peptide sequence having more proline residues than would be expected based on chance alone. In particular embodiments, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

The rigidity of protein linkers refers to the degree of flexibility of the protein backbone over the entire length of a short, single chain protein as measured by the average root-mean-square (RMS) (RMS^{fluct}) of all internal torsion angles (ϕ,ψ) over the length of a given single chain protein linker.

RMS^{fluct} of a torsion angle is the standard deviation of the torsion angle value about the time-averaged value in a CHARMm molecular dynamics simulation, wherein RMS^{fluct} is calculated as follows:

$${RMS}^{fluct} = \sqrt{\frac{1}{{N}_{f}} {\sum }_{f} {( {θ}^{f} - {θ}^{ave} )}^{2}}$$

where f refers to the frame number, N is the total number of frames in the trajectory file, and θ^{f} and θ^{ave} are the current value and the average value for the torsion angle, respectively.

“CHARMm” (Chemistry at HARvard Macromolecular Mechanics) refers to a computer simulation engine (see Brooks et al., (1983) J Comp Chem 4:187-217; MacKerell, et al., (1998) J. Phys. Chem. B 102(18): 3586-3616; and “CHARMM: The Energy Function and Its Parameterization with an Overview of the Program”, by MacKerell et al., in The Encyclopedia of Computational Chemistry, Volume 1, 271-277, by Paul von Raque Schleyer et al., editors (John Wiley & Sons: Chichester, United Kingdom (1998)); and Brooks, et al., (2009) J. Comp. Chem., 30:1545-1615 (2009).

In particular embodiments, the average RMS^{fluct} can be calculated using the formula: (average RMS^{fluct} phi (ϕ)+average RMS^{fluct} psi (ψ))/2. The average RMS fluctuation of all internal backbone torsion angles over the length of the protein can be used to quantify the rigidity of the protein linker. The more rigid the protein is the smaller the average RMS fluctuation should be due to a more limited conformational space accessible to the protein.

In particular embodiments, a rigid protein linker refers to a linker having an average RMS^{fluct} of 25 or less, 20 or less 15 or less when measured using CHARMm modeling over a production run of 200 picoseconds (ps). In particular embodiments, a semi-rigid protein linker refers to a linker having an average RMS^{fluct} of 45-25 when measured using CHARMm modeling over a production run of 200 picoseconds (ps).

As shown in FIG. 7, SARS-CoV-2 vaccine antigens can be configured to extend from the “top” and/or the “bottom” of a ring-like cTRP structure. Particular embodiments include different SARS-CoV-2 vaccine antigens extending from the top of a cTRP and from the bottom of the same cTRP (FIG. 7). In these “top” and/or the “bottom” embodiments, each “a” linker providing a SARS-CoV-2 vaccine antigen on the “top” of a cTRP and each “x” linker position providing a SARS-CoV-2 vaccine antigen on the “bottom” of a cTRP (or vice versa). Any portion of a linker loop can serve as the site for insertion of a SARS-CoV-2 vaccine antigen so long as the insertion does not impact the integrity of the flanking helices and the folding and function of the inserted SARS-CoV-2 vaccine antigen.

As indicated, SARS-CoV-2 vaccine antigens can be inserted within an “a” or “x” linker sequence. In particular embodiments, a SARS-CoV-2 vaccine antigen can replace an “a” or “x” linker sequence or can replace 1, 2, or 3 residues of an “a” or “x” linker sequence. In particular embodiments, the loops of interest at each position around the top or the bottom of a cTRP can be used as insertion sites for SARS-CoV-2 vaccine antigens in a variety of discrete ways, either by interrupting the loops internally (leaving the residues including the first positions and last positions of any loop flanking either side of the cargo) or by inserting adjacent to the loops.

In particular embodiments, the linker sequence is 2 amino acid residues and the SARS-CoV-2 vaccine antigen is inserted between the 2 residues. In particular embodiments, the linker sequence is 2 amino acid residues and the SARS-CoV-2 vaccine antigen replaces the 1st and/or the 2nd residue of the linker.

In particular embodiments, the linker sequence is 3 amino acid residues and the SARS-CoV-2 vaccine antigen is inserted N-terminally after the 1st residue of the linker sequence. In particular embodiments, the linker sequence is 3 amino acid residues and the SARS-CoV-2 vaccine antigen is inserted N-terminally after the 2nd residue of the linker sequence. In particular embodiments, the linker sequence is 3 amino acid residues and the SARS-CoV-2 vaccine antigen replaces the middle residue of the linker sequence.

In particular embodiments, the linker sequence is 5 amino acid residues and the SARS-CoV-2 vaccine antigen replaces the middle residue of linker. In particular embodiments, the linker sequence is 5 amino acid residues and the SARS-CoV-2 vaccine antigen is inserted between the 2nd and 3rd residues of the linker or the 3rd and 4th residues of the linker.

In particular embodiments, SARS-CoV-2 vaccine antigens can be directly inserted into cTRP sequences for expression and self-assembly. In particular embodiments, antibodies can be captured by SARS-CoV-2 vaccine antigens presented on a cTRP scaffold.

(iii) Recombinant Production. In particular embodiments, the SARS-CoV-2 vaccines disclosed herein are produced from a gene using a protein expression system. Protein expression systems can utilize DNA constructs (e.g., chimeric genes, expression cassettes, expression vectors, recombination vectors) including a nucleic acid sequence encoding the protein or proteins of interest operatively linked to appropriate regulatory sequences. In particular embodiments, such DNA constructs are not naturally-occurring DNA molecules and are useful for introducing DNA into host-cells to express selected proteins of interest. In particular embodiments, a DNA construct that encodes a vaccine protein can be inserted into cells (e.g., bacterial, mammalian, insect, etc.), which can produce the vaccine protein encoded by the DNA construct.

Operatively linked refers to the linking of DNA sequences (including the order of the sequences, the orientation of the sequences, and the relative spacing of the various sequences) in such a manner that the encoded protein is expressed. Methods of operatively linking expression control sequences to coding sequences are well known in the art. See, e.g., Maniatis et al., Molecular Cloning: A Laboratory Manual, Cold Spring Harbor, N. Y., 1982; and Sambrook et al., Molecular Cloning: A Laboratory Manual, Cold Spring Harbor, N. Y., 1989.

Expression control sequences are DNA sequences involved in any way in the control of transcription or translation. Suitable expression control sequences and methods of making and using them are well known in the art. Expression control sequences generally include a promoter. The promoter may be inducible or constitutive. It may be naturally-occurring, may be composed of portions of various naturally-occurring promoters, or may be partially or totally synthetic. Guidance for the design of promoters is provided by studies of promoter structure, such as that of Harley and Reynolds, Nucleic Acids Res., 15, 2343-2361, 1987. Also, the location of the promoter relative to the transcription start may be optimized. See, e.g., Roberts et al., Proc. Nati. Acad. Sci. USA, 76:760-764, 1979.

The promoter may include, or be modified to include, one or more enhancer elements. In particular embodiments, the promoter will include a plurality of enhancer elements. Promoters including enhancer elements can provide for higher levels of transcription as compared to promoters that do not include them.

For efficient expression, the coding sequences can be operatively linked to a 3′ untranslated sequence. In particular embodiments, the 3′ untranslated sequence can include a transcription termination sequence and a polyadenylation sequence. The 3′ untranslated region can be obtained, for example, from the flanking regions of genes.

In particular embodiments, a 5′ untranslated leader sequence can also be employed. The 5′ untranslated leader sequence is the portion of an mRNA that extends from the 5′ CAP site to the translation initiation codon.

In particular embodiments, a “hisavi” tag can be added to the N-terminus or C-terminus of a gene by the addition of nucleotides coding for the Avitag amino acid sequence, “GLNDIFEAQKIEWHE” (SEQ ID NO: 272), as well as the 6×histidine tag “HHHHHH” (SEQ ID NO: 273). The Avitag avidity tag can be biotinylated by a biotin ligase to allow for biotin-avidin or biotin-streptavidin based interactions for protein purification, as well as for immunobiology (such as immunoblotting or immunofluorescence) using anti-biotin antibodies. The 6×histidine tag allows for protein purification using Ni-²⁺ affinity chromatography.

In particular embodiments, SARS-CoV-2 vaccines on a cTRP scaffold can be produced using, for example, human suspension cells and/or the Daedalus expression system as described in Pechman et al., Am J Physiol 294: R1234-R1239, 2008. The Daedalus system utilizes inclusion of minimized ubiquitous chromatin opening elements in transduction vectors to reduce or prevent genomic silencing and to help maintain the stability of decigram levels of expression. This system can bypass tedious and time-consuming steps of other protein production methods by employing the secretion pathway of serum-free adapted human suspension cell lines, such as 293 Freestyle. Using optimized lentiviral vectors, yields of 20-100 mg/i of correctly folded and post-translationally modified, endotoxin-free protein of up to 70 kDa in size, can be achieved in conventional, small-scale (100 ml) culture. At these yields, most proteins can be purified using a single size-exclusion chromatography step, immediately appropriate for use in structural, biophysical or therapeutic applications. Bandaranayake et al., Nucleic Acids Res., 2011 (Nov); 39(21). In some instances, purification by chromatography may not be needed due to the purity of manufacture according the methods described herein.

In particular embodiments, the DNA constructs can be introduced by transfection, a technique that involves introduction of foreign DNA into the nucleus of eukaryotic cells. In particular embodiments, the proteins can be synthesized by transient transfection (DNA does not integrate with the genome of the eukaryotic cells, but the genes are expressed for 24-96 hours). Various methods can be used to introduce the foreign DNA into the host-cells, and transfection can be achieved by chemical-based means including by the calcium phosphate, by dendrimers, by liposomes, and by the use of cationic polymers. Non-chemical methods of transfection include electroporation, sono-poration, optical transfection, protoplast fusion, impalefection, and hydrodynamic delivery. In particular embodiments, transfection can be achieved by particle-based methods including gene gun where the DNA construct is coupled to a nanoparticle of an inert solid which is then “shot” directly into the target-cell's nucleus. Other particle-based transfection methods include magnet assisted transfection and impalefection.

Nucleic acid sequences encoding proteins disclosed herein can be derived by those of ordinary skill in the art. Nucleic acid sequences can also include one or more of various sequence polymorphisms, mutations, and/or sequence variants (e.g., splice variants or codon optimized variants). In particular embodiments, the sequence polymorphisms, mutations, and/or sequence variants do not affect the function of the encoded protein.

Sequence information provided by public databases can be used to identify additional gene and protein sequences that can be used with the systems and methods disclosed.

(iv) Compositions for Administration. SARS-CoV-2 therapeutics (protein vaccines, DNA or RNA-based vaccines, viral vectors, or cells modified to express a vaccine disclosed herein) can be formulated alone or in combination into compositions for administration to subjects. In particular embodiments, the SARS-CoV-2 therapeutics (e.g., SARS-CoV-2 vaccines) include immunogenic compositions. An immunogenic composition refers to an agent that stimulates an innate and/or an adaptive immune response in a subject.

Salts and/or pro-drugs of SARS-CoV-2 therapeutics can also be used.

A pharmaceutically acceptable salt includes any salt that retains the activity of the SARS-CoV-2 therapeutic and is acceptable for pharmaceutical use. A pharmaceutically acceptable salt also refers to any salt which may form in vivo as a result of administration of an acid, another salt, or a prodrug which is converted into an acid or salt.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids.

Suitable pharmaceutically acceptable base addition salts include metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine.

A prodrug includes an active ingredient which is converted to a therapeutically active compound after administration, such as by cleavage of a SARS-CoV-2 therapeutic or by hydrolysis of a biologically labile group.

In particular embodiments, compositions disclosed herein include a SARS-CoV-2 therapeutic of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts.

An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the SARS-CoV-2 therapeutic or helps to prevent denaturation or adherence to the container wall. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, α-monothioglycerol, and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on therapeutic weight.

The compositions disclosed herein can be formulated for administration by, for example, injection, inhalation, infusion, perfusion, lavage, or ingestion. The compositions disclosed herein can further be formulated for intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, intrathecal, intratumoral, intramuscular, intravesicular, and/or subcutaneous injection.

For injection, compositions can be formulated as aqueous solutions, such as in buffers including Hanks' solution, Ringer's solution, or physiological saline. The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as powders, capsules and tablets, suitable excipients include binders (gum tragacanth, acacia, cornstarch, gelatin), fillers such as sugars, e.g., lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insuffiator may also be formulated including a powder mix of SARS-CoV-2 therapeutic composition and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salts.

Additionally, compositions can be formulated as sustained-release systems utilizing semipermeable matrices of solid polymers including at least one SARS-CoV-2 therapeutic.

Various sustained-release materials have been established and are well known by those of ordinary skill in the art. Sustained-release systems may, depending on their chemical nature, release one or more SARS-CoV-2 therapeutics following administration for a few weeks up to over 100 days. Depot preparations can be administered by injection; parenteral injection; instillation; or implantation into soft tissues, a body cavity, or occasionally into a blood vessel with injection through fine needles.

Depot formulations can include a variety of bioerodible polymers including poly(lactide), poly(glycolide), poly(caprolactone) and poly(lactide)-co(glycolide) (PLG) of desirable lactide:glycolide ratios, average molecular weights, polydispersities, and terminal group chemistries. Blending different polymer types in different ratios using various grades can result in characteristics that borrow from each of the contributing polymers.

The use of different solvents (for example, dichloromethane, chloroform, ethyl acetate, triacetin, N-methyl pyrrolidone, tetrahydrofuran, phenol, or combinations thereof) can alter microparticle size and structure in order to modulate release characteristics. Other useful solvents include water, ethanol, dimethyl sulfoxide (DMSO), N-methyl-2-pyrrolidone (NMP), acetone, methanol, isopropyl alcohol (IPA), ethyl benzoate, and benzyl benzoate.

Exemplary release modifiers can include surfactants, detergents, internal phase viscosity enhancers, complexing agents, surface active molecules, co-solvents, chelators, stabilizers, derivatives of cellulose, (hydroxypropyl)methyl cellulose (HPMC), HPMC acetate, cellulose acetate, pluronics (e.g., F68/F127), polysorbates, Span® (Croda Americas, Wilmington, Delaware), poly(vinyl alcohol) (PVA), Brij® (Croda Americas, Wilmington, Delaware), sucrose acetate isobutyrate (SAIB), salts, and buffers.

Excipients that partition into the external phase boundary of microparticles such as surfactants including polysorbates, dioctylsulfosuccinates, poloxamers, PVA, can also alter properties including particle stability and erosion rates, hydration and channel structure, interfacial transport, and kinetics in a favorable manner.

Additional processing of the disclosed sustained release depot formulations can utilize stabilizing excipients including mannitol, sucrose, trehalose, and glycine with other components such as polysorbates, PVAs, and dioctylsulfosuccinates in buffers such as Tris, citrate, or histidine. A freeze-dry cycle can also be used to produce very low moisture powders that reconstitute to similar size and performance characteristics of the original suspension.

In certain examples, compositions include a vaccine adjuvant, an anti-infective agent, and/or a secondary vaccine. Examples of vaccine adjuvants, anti-infective agents, and secondary vaccines are described elsewhere herein.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

(v) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein.

Treating subjects includes delivering therapeutically effective amounts. Therapeutically effective amounts include those that provide effective amounts, prophylactic treatments and/or therapeutic treatments.

An “effective amount” is the amount of a composition necessary to result in a desired physiological change in the subject. For example, an effective amount can provide an immunogenic effect. Effective amounts are often administered for research purposes. Effective amounts disclosed herein can cause a statistically-significant effect in an in vitro assay, an animal model or clinical study relevant to the assessment of an infection's development, progression, and/or resolution, as well as the effects of the infection. An immunogenic composition can be provided in an effective amount, wherein the effective amount stimulates an immune response.

A “prophylactic treatment” includes a treatment administered to a subject who does not display signs or symptoms of an infection or displays only early signs or symptoms of an infection such that treatment is administered for the purpose of diminishing or decreasing the risk of developing the infection. Thus, a prophylactic treatment functions as a preventative treatment against an infection and/or the potential effects of an infection.

Particular uses of the compositions include use as prophylactic vaccines. Vaccines increase the immunity of a subject against a particular infection. Therefore, “SARS-CoV-2 vaccine” can refer to a treatment that increases the immunity of a subject against SARS-CoV-2.

In certain examples, a vaccine may be administered prophylactically, for example to a subject that is immunologically naive (e.g., no prior exposure or experience with SARS-CoV-2). In particular embodiments, a vaccine may be administered therapeutically to a subject who has been exposed to SARS-CoV-2. Thus, a vaccine can be used to ameliorate a symptom, syndrome and/or complication associated with SARS-CoV-2, examples of each of which are described elsewhere herein.

In particular embodiments, a SARS-CoV-2 vaccine is a therapeutically effective composition including one or more SARS-CoV-2 antigens that induces an immune response in a subject against SARS-CoV-2. The skilled artisan will appreciate that the immune system generally is capable of producing an innate immune response and an adaptive immune response. An innate immune response generally can be characterized as not being substantially antigen specific and/or not generating immune memory. An adaptive immune response can be characterized as being substantially antigen specific, maturing over time (e.g., increasing affinity and/or avidity for antigen), and in general can produce immunologic memory. Even though these and other functional distinctions between innate and adaptive immunity can be discerned, the skilled artisan will appreciate that the innate and adaptive immune systems can be integrated and therefore can act in concert.

In particular embodiments, administration of a SARS-CoV-2 vaccine can further include administration of one or more adjuvants. The term “adjuvant” refers to material that enhances the immune response to a vaccine antigen and is used herein in the customary use of the term. The precise mode of action is not understood for all adjuvants, but such lack of understanding does not prevent their clinical use for a wide variety of vaccines.

Exemplary vaccine adjuvants, include any kind of Toll-like receptor ligand or combinations thereof (e.g. CpG, Cpg-28 (a TLR9 agonist), polyriboinosinic polyribocytidylic acid (Poly(I:C)), α-galactoceramide, MPLA, Motolimod (VTX-2337, a novel TLR8 agonist developed by VentiRx), IMO-2055 (EMD1201081), TMX-101 (imiquimod), MGN1703 (a TLR9 agonist), G100 (a stabilized emulsion of the TLR4 agonist glucopyranosyl lipid A), Entolimod (a derivative of Salmonella flagellin also known as CBLB502), Hiltonol (a TLR3 agonist), and Imiquimod), and/or inhibitors of heat-shock protein 90 (Hsp90), such as 17-DMAG (17-dimethylaminoethylamino-17-demethoxygeldanamycin).

In particular embodiments a squalene-based adjuvant can be used. Squalene is part of the group of molecules known as triterpenes, which are all hydrocarbons with 30 carbon molecules. Squalene can be derived from certain plant sources, such as rice bran, wheat germ, amaranth seeds, and olives, as well as from animal sources, such as shark liver oil. In particular embodiments, the squalene-based adjuvant is MF599 (Novartis, Basel, Switzerland). An example of a squalene-based adjuvant that is similar to MF599 but is designed for preclinical research use is Addavax™ (InvivoGen, San Diego, CA). MF59 has been FDA approved for use in an influenza vaccine, and studies indicate that it is safe for use during pregnancy (Tsai T, et al. Vaccine. 2010. 17:28(7):1877-80; Heikkinen T, et al. Am J Obstet Gynecol. 2012. 207(3):177). In particular embodiments, squalene based adjuvants can include 0.1%-20% (v/v) squalene oil. In particular embodiments, squalene based adjuvants can include 5% (v/v) squalene oil.

In particular embodiments the adjuvant alum can be used. Alum refers to a family of salts that contain two sulfate groups, a monovalent cation, and a trivalent metal, such as aluminum or chromium. Alum is an FDA approved adjuvant. In particular embodiments, vaccines can include alum in the amounts of 1-1000 μg/dose or 0.1 mg-10 mg/dose.

In particular embodiments, one or more STING agonists are used as a vaccine adjuvant. “STING” is an abbreviation of “stimulator of interferon genes”, which is also known as “endoplasmic reticulum interferon stimulator (ERIS)”, “mediator of IRF3 activation (MITA)”, “MPYS” or “transmembrane protein 173 (TM173)”.

In particular embodiments, STING agonists include cyclic molecules with one or two phosphodiester linkages, and/or one or two phosphorothioate diester linkages, between two nucleotides. This includes (3′,5′)-(3′,5′) nucleotide linkages (abbreviated as (3′,3′)); (3′,5′)-(2′,5′) nucleotide linkages (abbreviated as (3′,2′)); (2′,5′)-(3′,5′) nucleotide linkages (abbreviated as (2′,3′)); and (2′,5′)-(2′,5′) nucleotide linkages (abbreviated as (2′,2′)). “Nucleotide” refers to any nucleoside linked to a phosphate group at the 5′, 3′ or 2′ position of the sugar moiety.

In particular embodiments, STING agonists include c-AIMP; (3′,2′)c-AIMP; (2′,2′)c-AIMP; (2′,3′)c-AIMP; c-AIMP(S); c-(dAMP-dIMP); c-(dAMP-2′FdIMP); c-(2′FdAMP-2′FdIMP); (2′,3′)c-(AMP-2′FdlMP); c-[2′FdAMP(S)-2′FdIMP(S)]; c-[2′FdAMP(S)-2′FdlMP(S)](POM)2; and DMXAA. Additional examples of STING agonists are described in WY02016/145102.

Other immune stimulants can also be used as vaccine adjuvants. Additional exemplary small molecule immune stimulants include TGF-β inhibitors, SHP-inhibitors, STAT-3 inhibitors, and/or STAT-5 inhibitors. Exemplary siRNA capable of down-regulating immune-suppressive signals or oncogenic pathways (such as kras) can be used whereas any plasmid DNA (such as minicircle DNA) encoding immune-stimulatory proteins can also be used.

In particular embodiments, the immune stimulant may be a cytokine and or a combination of cytokines, such as IL-2, IL-12 or IL-15 in combination with IFN-α, IFN-s or IFN-γ, or GM-CSF, or any effective combination thereof, or any other effective combination of cytokines. The above-identified cytokines stimulate T_{H}1 responses, but cytokines that stimulate T_{H}2 responses may also be used, such as IL-4, IL-10, IL-11, or any effective combination thereof. Also, combinations of cytokines that stimulate T_{H}1 responses along with cytokines that stimulate T_{H}2 responses may be used.

“Immune response” refers to a response of the immune system to a SARS-CoV-2 antigen disclosed herein. In particular embodiments, an immune response to a SARS-CoV-2 antigen can be an innate and/or adaptive response. In particular embodiments, an adaptive immune response can be a “primary immune response” which refers to an immune response occurring on the first exposure of a “naive” subject to a SARS-CoV-2 antigen. For example, in the case of a primary antibody response, after a lag or latent period of from 3 to 14 days depending on, for example, the composition, dose, and subject, antibodies to the SARS-CoV-2 antigen can be produced. Generally, IgM production lasts for several days followed by IgG production and the IgM response can decrease. Antibody production can terminate after several weeks but memory cells can be produced. In particular embodiments, an adaptive immune response can be a “secondary immune response”, “anamnestic response,” or “booster response” which refer to the immune response occurring on a second and subsequent exposure of a subject to a SARS-CoV-2 antigen disclosed herein. Generally, in a secondary immune response, memory cells respond to the SARS-CoV-2 antigen and therefore the secondary immune response can differ from a primary immune response qualitatively and/or quantitatively. For example, in comparison to a primary antibody response, the lag period of a secondary antibody response can be shorter, the peak antibody titer can be higher, higher affinity antibody can be produced, and/or antibody can persist for a greater period of time.

In particular embodiments, an immune response against SARS-CoV-2 will include antibody production against: the S protein, the E protein or the M protein. In particular embodiments, an immune response will include antibody production against the administered antigen (e.g, SEQ ID NO: 1 or 5 or sequences having at least 90% sequence identity to SEQ ID NO: 1 or 5).

A “therapeutic treatment” includes a treatment administered to a subject who displays symptoms or signs of an infection and is administered to the subject for the purpose of diminishing or eliminating those signs or symptoms of the infection or effects of the infection. The therapeutic treatment can reduce, control, or eliminate the presence or activity of the infection and/or reduce, control or eliminate side effects of the infection.

In particular embodiments a therapeutic treatment can reduce, control, or eliminate a primary infection with SARS-CoV-2. In particular embodiments a therapeutic treatment can reduce or eliminate the symptoms of SARS-CoV-2.

In particular embodiments, a therapeutically effective amount reduces or prevents transmission of SARS-CoV2.

In particular embodiments, a therapeutically effective amount alleviates or reduces the severity or occurrence of symptoms, syndromes, and/or complications associated with SARS-CoV2 infection. Exemplary symptoms include fever, severe headache, muscle pain, malaise, extreme asthenia, conjunctivitis, popular rash, dysphagia, nausea, vomiting, bloody diarrhea followed by diffuse hemorrhages, delirium, shock, jaundice, thrombocytopenia, lymphocytopenia, neutrophilia, focal necrosis in various organs (e.g., kidneys and liver), and acute respiratory distress. Exemplary syndromes include 2019 novel coronavirus-infected pneumonia (NCIP), acute lung injury (ALI), and acute respiratory distress syndrome (ARDS). Exemplary complications include hypoxemic respiratory failure, acute respiratory failure (ARF), acute liver injury, acute cardiac injury, acute kidney injury, septic shock, disseminated intravascular coagulation, blood clots, multisystem inflammatory syndrome, chronic fatigue, rhabdomyolysis, and cytokine storm.

In particular embodiments, a therapeutically effective amount results in a reduction in the incidence of progressive respiratory insufficiency (PRI) as measured by greater than or equal to a 1-tier or even a 2-tier or more change in respiratory support methods required to maintain satisfactory oxygenation (Sp02>93%) using the 6-tier hierarchical levels of respiratory support methods described below, wherein the subject has acquired a SARS-CoV2 infection but has previously been, or will be, administered a SARS-CoV-2 vaccine disclosed herein.

The scale of increasing respiratory support levels includes:
Level 1: Normal oxygenation on room air (Sp02>93%), no need for supplemental 02;
Level 2: Persistent hypoxemia on room air (Sp02>93) with requirement for low-level supplemental 02 by nasal cannular or mask (up to 2/min) to maintain Sp02>93;
Level 3: Requirement for higher levels of passive supplemental 02 by nasal cannular or mask (up to 2 L/min) to maintain Sp02>93;
Level 4: Requirement for oxygenation by positive-pressure devices, e.g., Continuous Positive Airway Pressure (CPAP) or Bi-level Positive Airway Pressure (BiPAP) or other non-invasive positive-pressure respiratory support methods to main satisfactory oxygenation and/or ventilation;
Level 5: Requires invasive respiratory support (intubated mechanical ventilation or ECMO);
Level 6: Death.

In particular embodiments, the reduction in PRI is a change from level 5 to level 3, level 5 to level 2, or level 5 to level 1. In one embodiment, the reduction in PRI is a change from level 4 to level 2 or level 4 to level 1. In one embodiment, the reduction in PRI is a change from level 3 to level 1.

In particular embodiments, a therapeutically effective amount reduces the median time to Clinical Recovery (status 6, 7, or 8 in the NIAID Clinical Status scale using an adapted National Institute of Allergy and Infectious Diseases (NIAID) ordinal scale of Clinical Status) by at least 3, 4, 5, or more days. In particular embodiments, a therapeutically effective amount results in an improvement as measured by the adapted ordinal scale of Clinical Status in a subject that develops a SARS-CoV2 infection.

From most severe disease to progressively less severe disease, the stages of the adapted ordinal scale of overall Clinical Status are defined as follows:
1. Death;
2. Hospitalized, on invasive mechanical ventilation or ECMO;
3. Hospitalized, on non-invasive ventilation or high flow oxygen devices;
4. Hospitalized, requiring supplemental oxygen;
5. Hospitalized, not requiring supplemental oxygen—requiring ongoing medical care (COVID-19 related or otherwise);
6. Hospitalized, not requiring supplemental oxygen; no longer requires close medical care for COVID-19;
7. Not hospitalized, but with limitation on activities and needing close outpatient care for COVID-19 manifestations;
8. Not hospitalized, no limitations on activities, no need for continued close medical care.

In particular embodiments, a therapeutically effective amount reduces the median time to Clinical Recovery (status 6, 7, or 8 in the NIAID Clinical Status scale using an adapted National Institute of Allergy and Infectious Diseases (NIAID) ordinal scale of Clinical Status) by at least 5 days, at least 6 days, at least 7 days, at least 8 days, at least 9 days, or at least 10 days as compared to a subject that has not received a SARS-CoV-2 vaccine disclosed herein.

In particular embodiments, a therapeutically effective amount reduces the duration of hospitalization for a subject infected with the SARS-CoV-2 virus as compared to a subject that has not received a SARS-CoV-2 vaccine disclosed herein.

In particular embodiments, a therapeutically effective amount reduces the time to sustained non-detectable SARS-CoV-2 virus in the nose and/or throat in a patient infected with the SARS-CoV-2 virus as compared to a subject that has not received a SARS-CoV-2 vaccine disclosed herein.

In particular embodiments, a therapeutically effective amount reduces respiratory failure or death as compared to a subject that has not received a SARS-CoV-2 vaccine disclosed herein.

Function as an effective amount, prophylactic treatment or therapeutic treatment are not mutually exclusive, and in particular embodiments, administered dosages may accomplish more than one treatment type.

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of infection, stage of infection, effects of infection (e.g., IM, lymphoproliferative disorders), previous or concurrent therapeutic interventions, idiopathy of the subject and route of administration.

Useful doses can range from 0.1 to 5 μg/kg or from 0.5 to 1 μg/kg. In other non-limiting examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other non-limiting examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

In some embodiments, the SARS-CoV-2 vaccines described herein can be administered in combination or alternation with a secondary SARS-CoV2 vaccine. In some embodiments, the secondary vaccine can be selected from an mRNA-based vaccine, an adenovirus vaccine, a non-replicating vaccine, a DNA vaccine, a live attenuated vaccine, a plant-based adjuvant vaccine, a multipope peptide-based vaccine, an inactivated virus, and a peptide vaccine, or combinations thereof. Additional exemplary secondary vaccines suitable for use with the SARS-CoV-2 vaccines and methods described herein include mRNA-1273 (MODERNA COVID-19 VACCINE; Modema, Inc.), AZD-1222 (COVIDSEQELD; AstraZeneca and University of Oxford), BNT162 (COMIRNATY; Pfizer and BioNTech), Sputnik V (Gamaleya Research Institute, Acellena Contract Drug Research and Development), CoronaVac (Sinovac), NVX-CoV 2372 (NovoVax), SCB-2019 (Sanofi and GSK), ZyCoV-D (Zydus Cadila), BBIBP-CorV (Beijing Institute of Biological Products; China National Pharmaceutical Group (Sinopharm)), EpiVacCorona (Federal Budgetary Research Institution State Research Center of Virology and Biotechnology), Convidicea (CanSino Biologies), Covid-19 Vaccine (Wuhan Institute of Biological Products; China National Pharmaceutical Group (Sinopharm), JNJ-78436735 (Johnson & Johnson), ZF2001) Anhui Zhifei Longcom Biopharmaceutical, Institute of Microbiology of the Chinese Academy of Sciences), CVnCoV (CureVac; GSK), INO-4800 (Inovio Pharmaceuticals), VIR-7831 (Medicago; GSK; Dynavax), Covid-19 adenovirus bases vaccine (ImmunityBio; NantKwest), UB-612 (COVAXX), CoVaxin (Bharat Biotech), or combinations thereof.

In some embodiments, SARS-CoV-2 vaccines disclosed herein can be used as a booster vaccine, to increase or modify or alter immune responses induced by a prior SARS-CoV2 vaccine; such as an RNA vaccine, a DNA vaccine, a viral vector vaccine, or a protein-based vaccine. In some embodiments, the SARS-CoV-2 vaccines disclosed herein can be used as a booster vaccine following infection and recovery from SARS-CoV2.

The SARS-CoV-2 vaccines described herein can be administered on top of the current standard of care for COVID patients, or in combination or alternation with any other compound or therapy that the healthcare provider deems beneficial for the patient. The combination and/or alternation therapy can be therapeutic, adjunctive, or palliative.

In some embodiments, the SARS-CoV-2 vaccines is administered with an anti-infective agent, for example a NSSB inhibitor including remdesivir, or a protease inhibitor such as lopinavir or ritonavir. In some embodiments, the anti-infective is selected from favipiravir, fmgolimod (Gilenya), methylprednisolone, bevacizumab (Avastin), Actemra (tocilizumab), umifenovir, losartan and the monoclonal antibody combination of REGN3048 and REGN3051, ribavirin, AT-527 (Atea Pharmaceuticals, described in U.S. Pat. No. 10,519,186) or AT-511 (Atea Pharmaceuticals, described in U.S. Pat. No. 10,519,186). Any of these drugs or vaccines can be used in combination or alternation with the SARS-CoV-2 vaccines provided herein to treat a SARS-CoV2 viral infection.

The pharmaceutical compositions described herein can be administered by, without limitation, injection, inhalation, infusion, perfusion, lavage or ingestion. Routes of administration can include intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual administration and more particularly by intravenous, intradermal, intraarterial, intraparenteral, intranasal, intranodal, intralymphatic, intraperitoneal, intralesional, intraprostatic, intravaginal, intrarectal, topical, intrathecal, intratumoral, intramuscular, intravesicular, oral, subcutaneous, and/or sublingual injection.

(vi) Kits. Also disclosed herein are kits including one or more containers including one or more of the SARS-CoV-2 vaccines (in protein or nucleic acid form), modified cells (e.g. cells modified to express a vaccine disclosed herein), and/or compositions and/or adjuvants, anti-infective agents, or secondary vaccines described herein. Associated with such container(s) can be a notice in the form prescribed by a governmental agency regulating the manufacture, use, or sale of pharmaceuticals or biological products, which notice reflects approval by the agency of manufacture, use, or sale for human administration.

(vii) Exemplary Embodiments.
1. A SARS-CoV-2 vaccine including the sequence as set forth in SEQ ID NO: 4 or 9 or a sequence having at least 90% sequence identity to the sequence as set forth in SEQ ID NO: 4 or 9.
2. The SARS-CoV-2 vaccine of embodiment 1, having 3 copies of SEQ ID NO: 4 or 3 copies of sequences having at least 90% sequence identity to the sequence as set forth in SEQ ID NO: 4, wherein the 3 copies self-assemble to result in trimeric presentation of the sequence as set forth in SEQ ID NO: 1 or a sequence having at least 90% sequence identity to the sequence as set forth in SEQ ID NO: 1.
3. The SARS-CoV-2 vaccine of embodiment 1 or 2, having 4 copies of SEQ ID NO: 9 or 4 copies of sequences having at least 90% sequence identity to the sequence as set forth in SEQ ID NO: 9, wherein the 4 copies self-assemble to result in tetrameric presentation of the sequence as set forth in SEQ ID NO: 5 or a sequence having at least 90% sequence identity to the sequence as set forth in SEQ ID NO: 5.
4. The SARS-CoV-2 vaccine of any of embodiments 1-3, including the sequence as set forth in SEQ ID NO: 3 or 8 or a sequence having at least 90% sequence identity to the sequence as set forth in SEQ ID NO: 3 or 8.
5. The SARS-CoV-2 vaccine of any of embodiments 1-4, the sequence as set forth in SEQ ID NO: 2 or 7 or a sequence having at least 90% sequence identity to the sequence as set forth in SEQ ID NO: 2 or 7.
6. A SARS-CoV-2 vaccine including a circular, handed protein including at least three α-helical structures wherein each α-helical structure includes an outer α helix and an inner α helix joined by a flexible linker and wherein each α-helical structure has at least 95% sequence identity with an adjacent α-helical structure.
7. The SARS-CoV-2 vaccine of embodiment 6, having the formula: (d-a-b-x-y)n, (a-d-b-x-y)n, (a-b-d-x-y)n, (a-b-x-d-y)n or (a-b-x-y-d)n wherein
   b and y each represent a linker,
   a represents an amino acid sequence that forms an alpha (α) helix,
   x represents an amino acid sequence that forms a second α helix,
   d represents an SARS-CoV-2 vaccine antigen, and
   the protein is handed.
8. The SARS-CoV-2 vaccine of embodiment 6, having the formula: (d-a-b-x-y)n, (a-d-b-x-y)n, (a-b-d-x-y)n, (a-b-x-d-y)n or (a-b-x-y-d)n wherein
   a and x each represent a linker,
   b represents an amino acid sequence that forms an alpha (α) helix,
   y represents an amino acid sequence that forms a second α helix,
   d represents an SARS-CoV-2 vaccine antigen, and
   the protein is handed.
9. The SARS-CoV-2 vaccine of embodiment 8, wherein n=3, 4, 6, 9, 12, or 24.
10. The SARS-CoV-2 vaccine of embodiment 8 or 9, wherein each b and y segment has 13 amino acid residues.
11. The SARS-CoV-2 vaccine of any of embodiments 8-10, wherein each a and x linker represent a 2, 3, 4, or 5 amino acid linker.
12. The SARS-CoV-2 vaccine of any of embodiments 8-11, wherein the N-terminal b segment has 13 amino acids and a cysteine at position 1 and the C-terminal segment b segment has 13 amino acids and a cysteine at position 3.
13. The SARS-CoV-2 vaccine of embodiment 12, wherein the N-terminal b segment has the sequence as set forth in SEQ ID NO: 80, 81, 91, 92, 93, 94, 95, 96, 97, 98, 99, 100, or 101.
14. The SARS-CoV-2 vaccine of embodiment 12, wherein the N-terminal b segment has the sequence as set forth in SEQ ID NO: 95.
15. The SARS-CoV-2 vaccine of any of embodiments 8-14, wherein the C-terminal b segment has the sequence as set forth in SEQ ID NO. 80, 81, 89, 91, 92, 93, 94, 99, 103, 104, or 105.
16. The SARS-CoV-2 vaccine of any of embodiments 8-15, wherein the C-terminal b segment has the sequence as set forth in SEQ ID NO: 89.
17. The SARS-CoV-2 vaccine of any of embodiments 8-16, including the sequence as set forth in SEQ ID NO: 6 or 291 or having at least 90% sequence identity to the sequence as set forth in SEQ ID NO: 6 or 291.
18. The SARS-CoV-2 vaccine of any of embodiments 8-17, wherein the SARS-CoV-2 vaccine antigen includes the S protein or fragment thereof.
19. The SARS-CoV-2 vaccine of any of embodiments 8-18, wherein the SARS-CoV-2 vaccine antigen includes the SI+S2 fragment of the S protein
20. The SARS-CoV-2 vaccine of any of embodiments 8-19, wherein the SARS-CoV-2 vaccine antigen includes the receptor binding domain (RBD) of the S protein.
21. The SARS-CoV-2 vaccine of any of embodiments 8-20, wherein the SARS-CoV-2 vaccine antigen includes residues 327 to 524 of the S protein, residues 331 to 524 of the S protein, residues 504 to 524 of the S protein, or residues 473 to 490 of the S protein.
22. The SARS-CoV-2 vaccine of any of embodiments 8-21, wherein the SARS-CoV-2 vaccine antigen has the sequence as set forth in SEQ ID NO: 1 or 5 or has a sequence with at least 90% sequence identity to the sequence as set forth in SEQ ID NO: 1 or 5.
23. The SARS-CoV-2 vaccine of any of embodiments 9-22, wherein n=3, and the SARS-CoV-2 vaccine has 3 copies of the sequence as set forth in SEQ ID NO: 1 or 3 copies of sequences having at least 90% sequence identity to the sequence as set forth in SEQ ID NO: 1.
24. The SARS-CoV-2 vaccine of any of embodiments 9-22, wherein n=4, and the SARS-CoV-2 vaccine has 4 copies of the sequence as set forth in SEQ ID NO: 5 or 4 copies of sequences having at least 90% sequence identity to the sequence as set forth in SEQ ID NO: 5.
25. The SARS-CoV-2 vaccine of any of embodiments 18-24, wherein the S protein or fragment thereof has substitutions or deletions selected from
K417T, E484K or N501Y;
K417T, E484K, and N501Y;
K417N;
D614G, A570D, P681H, T716I, S982A, D1118H, K417N or K417T;
D215G, A701V, L18F, R2461, Y453F, 1692V, M1229I, N439K, A222V, S477N, or A376T;
deletion of one or more residues H69, V70, or Y144;
deletion of residues 242-244;
N501Y, A570D, D614G, P681H, T716I, S982A, and D1118H, deletion of residues 69-70, and deletion of residue Y144;
N501Y, K417N, K417T, E484K, D80A, A701V, and L18F and deletion of residues 242-244;
N439K, T478I, S477N, P479S, V367F, V341I, P330S, P384L, and A522V; or
D614G, D936Y, P1263L, L5F, N439K, R21I, D839Y, L54F, A879S, L18F, F1121L, R847K, T478I, A829T, Q675H, S477N, H49Y, T29I, G769V, G1124V, V1176F, K1073N, P479S, S1252P, Y145, E583D, R214L, A1020V, Q1208H, D215G, H146Y, S98F, T95I, G1219C, A846V, 1197V, R102I, V367F, T572I, A1078S, A831V, P1162L, T73I, A845S, G1219V, H245Y, L8V, Q675R, S254F, V483A, Q677H, D138H, D80Y, M1237T, D1146H, E654D, H655Y, S50L, S939F, S943P, G485R, Q613H, T76I, V341I, M153I, S221L, T859I, W258L, L242F, P681L, V289I, A520S, V1104L, V1228L, L176F, M1237I, T307I, T716I, L14I, M1229I, A1087S, P26S, P330S, P384L, R765L, S940F, T323I, V826L, E1202Q, L1203F, L611F, V615I, A262S, A522V, A688V, A706V, A892S, E554D, 0836H, T1027I, T22I, A222V, A27S, A626V, C1247F, K1191N, M731I, P26L, S1147L, S1252F, S255F, V1264L, V308L, D80A, 1670L, P251L, P631S, *1274Q, A344S, A771S, A879T, D1084Y, D253G, H1101Y, L1200F, Q14H, Q239K, A623V, D215Y, E1150D, G476S, K77M, M177I, P812S, S704L, T51I, T547I, T791I, V1122L, Y145H, D574Y, G142D, G181V, 1834T, N370S, P812L, S12F, T791P, V90F, W152L, A292S, A570V, A647S, A845V, D1163Y, G181R, L84I, L938F, P1143L, P809S, R78M, T1160I, V1133F, V213L, V615F, A831V, D839Y, D839N, D839E, S943P, P1263L, or V622F.
26. The SARS-CoV-2 vaccine of any of embodiments 8-25, wherein b and y have at least 98% sequence identity.
27. The SARS-CoV-2 vaccine of any of embodiments 8-25, wherein the linkers are flexible linkers.
28. The SARS-CoV-2 vaccine of embodiment 27, wherein the flexible linkers include GBB linkers.
29. The SARS-CoV-2 vaccine of embodiment 27, wherein the flexible linkers have the sequence GD, GN, GS, GT, GLD, GLN, GLS, GLT, GID, GIN, GIT, GIS, GVD, GVN, GVT, GVS, GTD, GTN, GTT, GTS, GKS, GYS, GNS, LPHD (SEQ ID NO: 274), NPND (SEQ ID NO: 275), DPKD (SEQ ID NO: 276), GLEPD (SEQ ID NO: 277), GVSLD (SEQ ID NO: 278), or GVLPD (SEQ ID NO: 279).
30. The SARS-CoV-2 vaccine of embodiment 27, wherein the flexible linkers have the sequence GLD, GLN, GLS, GLT, GID, GIN, GIT, GIS, GVD, GVN, GVT, GVS, GTD, GTN, GTT, GTS, GKS, GYS, or GNS.
31. The SARS-CoV-2 vaccine of embodiment 27, wherein the flexible linkers have the sequence GKS; GIT; GTT; GYS; GDK; GDE; NDK; GDR; GDL; or GIS.
32. The SARS-CoV-2 vaccine of any of embodiments 8-31, having at least two SARS-CoV-2 vaccine antigens wherein each SARS-CoV-2 vaccine antigen is inserted into the sequence of the protein between an outer α helix of the protein and an adjacent inner α helix of the protein.
33. The SARS-CoV-2 vaccine of embodiment 32, wherein at least one of the SARS-CoV-2 vaccine antigens has the sequence as set forth in SEQ ID NO: 1 or 5 or has a sequence with at least 90% sequence identity to the sequence as set forth in SEQ ID NO: 1 or 5.
34. The SARS-CoV-2 vaccine of any of embodiments 8-33, further including a SARS-CoV-2 vaccine antigen (d) that replaces 1, 2, or 3 residues of an a or x linker sequence.
35. The SARS-CoV-2 vaccine of any of embodiments 8-34, further including at least two SARS-CoV-2 vaccine antigens (d) inserted in a (a-b-x-y) unit within or adjacent to an a or x linker sequence.
36. The SARS-CoV-2 vaccine of any of embodiments 8-35, further including a flexible, rigid, or semi-rigid linker adjacent to a SARS-CoV-2 vaccine antigen.
37. The SARS-CoV-2 vaccine of embodiment 36, wherein the linker is a flexible linker.
38. The SARS-CoV-2 vaccine of embodiment 37, wherein the linker has the sequence GS or the sequence as set forth in SEQ ID NO: 269 or 292.
39. A nucleotide sequence encoding a SARS-CoV-2 vaccine of any of embodiments 1-38.
40. A cell including a nucleotide sequence of embodiment 39.
41. A composition formulated for administration to a subject including a SARS-CoV-2 vaccine of any of embodiments 1-38, a nucleotide sequence of embodiment 39, or a cell of embodiment 40.
42. The composition of embodiment 41, further including an adjuvant.
43. The composition of embodiment 42, wherein the adjuvant includes alum, a squalene-based adjuvant, a STING agonist, or a liposome-based adjuvant.
44. The composition of any of embodiments 41-43, further including anti-infective agent.
45. The composition of embodiment 44, wherein the anti-infective agent includes a NSSB inhibitor or a protease inhibitor.
46. The composition of embodiment 44, wherein the anti-infective agent includes remdesivir, lopinavir, ritonavir, favipiravir, fmgolimod, methylprednisolone, bevacizumab, tocilizumab, umifenovir, losartan, the monoclonal antibody combination of REGN3048 and REGN3051, ribavirin, AT-527 or AT-511.
47. The composition of any of embodiments 41-46, wherein the composition further includes a second vaccine including mRNA-1273, AZD-1222, BNT162, Sputnik V, CoronaVac, NVX-CoV 2372, SCB-2019, ZyCoV-D, BBIBP-CorV, EpiVacCorona, Convidicea, Covid-19 Vaccine, JNJ-78436735, ZF2001, CVnCoV, INO-4800, VIR-7831, Covid-19 adenovirus bases vaccine, UB-612, or CoVaxin.
48. A method of stimulating an anti-SARS-CoV-2 immune response in a subject including administering to the subject a therapeutically effective amount of a composition of embodiment 37 to the subject, thereby stimulating a SARS-CoV-2 immune response in the subject.
49. The method of embodiment 48, wherein the subject is a SARS-CoV-2 seronegative subject.
50. The method of embodiment 48, wherein the subject is a SARS-CoV-2 seropositive subject.
51. The method of any of embodiments 48-50, wherein the method results in antibody production against the SARS-CoV-2 antigen of the vaccine.
52. The method of any of embodiments 48-51, wherein the administering precedes or follows administration of a different SARS-CoV-2 vaccine.
53. The method of embodiment 52, wherein the different SARS-CoV-2 vaccine includes mRNA-1273, AZD-1222, BNT162, Sputnik V, CoronaVac, NVX-CoV 2372, SCB-2019, ZyCoV-D, BBIBP-CorV, EpiVacCorona, Convidicea, Covid-19 Vaccine, JNJ-78436735, ZF2001, CVnCoV, INO-4800, VIR-7831, Covid-19 adenovirus bases vaccine, UB-612, or CoVaxin.
54. The method of any of embodiments 48-53, further including administering an adjuvant to the subject.
55. The method of embodiment 54, wherein the adjuvant includes alum, a squalene-based adjuvant, a STING agonist, or a liposome-based adjuvant.
56. The method of any of embodiments 48-55, further including administering an anti-infective agent to the subject.
57. The method of embodiment 56, wherein the anti-infective agent includes a NSSB inhibitor or a protease inhibitor.
58. The method of embodiment 56, wherein the anti-infective agent includes remdesivir, lopinavir, ritonavir, favipiravir, fmgolimod, methylprednisolone, bevacizumab, tocilizumab, umifenovir, losartan, the monoclonal antibody combination of REGN3048 and REGN3051, ribavirin, AT-527 or AT-511.
59. The method of any of embodiments 48-58, wherein the method treats the subject for a symptom, syndrome, or complication of SARS-CoV-2 infection.
60. The method of embodiment 59, wherein the symptom of SARS-CoV-2 infection includes fever, headache, muscle pain, malaise, asthenia, conjunctivitis, rash, dysphagia, nausea, vomiting, bloody diarrhea, delirium, shock, jaundice, thrombocytopenia, lymphocytopenia, neutrophilia, focal necrosis in organs, or acute respiratory distress.
61. The method of embodiment 59, wherein the syndrome of SARS-CoV-2 infection includes 2019 novel coronavirus-infected pneumonia (NCIP), acute lung injury (ALI), or acute respiratory distress syndrome (ARDS).
62. The method of embodiment 59, wherein the complication of SARS-CoV-2 infection includes hypoxemic respiratory failure, acute respiratory failure (ARF), acute liver injury, acute cardiac injury, acute kidney injury, septic shock, disseminated intravascular coagulation, blood clots, multisystem inflammatory syndrome, chronic fatigue, rhabdomyolysis, or cytokine storm.
63. The method of any of embodiments 48-62, wherein the method results in a reduction in the incidence of progressive respiratory insufficiency (PRI).

(viii) Closing Paragraphs. As indicated previously, variants of the sequences disclosed and referenced herein are included. In particular embodiments, variants of proteins can include those having one or more conservative amino acid substitutions or one or more non-conservative substitutions that do not adversely affect the function of the protein in a relevant physiological measure. A “conservative substitution” involves a substitution found in one of the following conservative substitutions groups: Group 1: Alanine (Ala), Glycine (Gly), Serine (Ser), Threonine (Thr); Group 2: Aspartic acid (Asp), Glutamic acid (Glu); Group 3: Asparagine (Asn), Glutamine (Gln); Group 4: Arginine (Arg), Lysine (Lys), Histidine (His); Group 5: Isoleucine (lie), Leucine (Leu), Methionine (Met), Valine (Val); and Group 6: Phenylalanine (Phe), Tyrosine (Tyr), Tryptophan (Trp).

Additionally, amino acids can be grouped into conservative substitution groups by similar function or chemical structure or composition (e.g., acidic, basic, aliphatic, aromatic, sulfur-containing). For example, an aliphatic grouping may include, for purposes of substitution, Gly, Ala, Val, Leu, and lie. Other groups containing amino acids that are considered conservative substitutions for one another include: sulfur-containing: Met and Cysteine (Cys); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, lie, Val, and Cys; and large aromatic residues: Phe, Tyr, and Trp. Additional information is found in Creighton (1984) Proteins, W.H. Freeman and Company.

In particular embodiments, variants of the protein sequences (e.g., vaccine proteins, and/or cTRP scaffolds) disclosed herein include sequences with at least 70% sequence identity, at least 80%, at least 85%, at least 90%, at least 95%, at least 96%, at least 97%, at least 98%, or at least 99% sequence identity to the protein sequences described or disclosed herein.

“% sequence identity” refers to a relationship between two or more sequences, as determined by comparing the sequences. In the art, “identity” also means the degree of sequence relatedness between protein sequences or nucleic acid sequences as determined by the match between strings of such sequences. “Identity” (often referred to as “similarity”) can be readily calculated by known methods, including (but not limited to) those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine % sequence identity are designed to give the best match between the sequences tested. Methods to determine % sequence identity and similarity are codified in publicly available computer programs. Sequence alignments and % sequence identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992,111-20. Editor(s): Suhai, Sandor. Publisher: Plenum, New York, NY. Within the context of this disclosure it will be understood that where sequence analysis software is used for analysis, the results of the analysis are based on the “default values” of the program referenced. “Default values” will mean any set of values or parameters, which originally load with the software when first initialized.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. In particular embodiments, a material effect would cause a statistically-significant reduction in a primary antibody response to a SARS-CoV-2 vaccine disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein.

Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. A SARS-CoV-2 vaccine comprising the sequence as set forth in SEQ ID NO: 4 or 9 or a sequence having at least 98% sequence identity to the sequence as set forth in SEQ ID NO: 4 or 9.

2. The SARS-CoV-2 vaccine of claim 1, having 3 copies of SEQ ID NO: 4 or 3 copies of sequences having at least 98% sequence identity to the sequence as set forth in SEQ ID NO: 4, wherein the 3 copies self-assemble to result in trimeric presentation of the sequence as set forth in SEQ ID NO: 1 or a sequence having at least 98% sequence identity to the sequence as set forth in SEQ ID NO: 1.

3. The SARS-CoV-2 vaccine of claim 1, having 4 copies of SEQ ID NO: 9 or 4 copies of sequences having at least 98% sequence identity to the sequence as set forth in SEQ ID NO: 9, wherein the 4 copies self-assemble to result in tetrameric presentation of the sequence as set forth in SEQ ID NO: 5 or a sequence having at least 98% sequence identity to the sequence as set forth in SEQ ID NO: 5.

4. The SARS-CoV-2 vaccine of claim 1, comprising the sequence as set forth in SEQ ID NO: 3 or 8 or a sequence having at least 98% sequence identity to the sequence as set forth in SEQ ID NO: 3 or 8.

5. The SARS-CoV-2 vaccine of claim 1, the sequence as set forth in SEQ ID NO: 2 or 7 or a sequence having at least 98% sequence identity to the sequence as set forth in SEQ ID NO: 2 or 7.

6-40. (canceled)

41. A composition formulated for administration to a subject including a SARS-CoV-2 vaccine of claim 1.

42. The composition of claim 41, further comprising an adjuvant.

43. The composition of claim 42, wherein the adjuvant comprises alum, a squalene-based adjuvant, a STING agonist, or a liposome-based adjuvant.

44. The composition of claim 41, further comprising anti-infective agent.

45. The composition of claim 44, wherein the anti-infective agent comprises a NS5B inhibitor or a protease inhibitor.

46. The composition of claim 44, wherein the anti-infective agent comprises remdesivir, lopinavir, ritonavir, favipiravir, fmgolimod, methylprednisolone, bevacizumab, tocilizumab, umifenovir, losartan, the monoclonal antibody combination of REGN3048 and REGN3051, ribavirin, AT-527 or AT-511.

47. The composition of claim 41, wherein the composition further comprises a second vaccine comprising mRNA-1273, AZD-1222, BNT162, Sputnik V, CoronaVac, NVX-CoV 2372, SCB-2019, ZyCoV-D, BBIBP-CorV, EpiVacCorona, Convidicea, Covid-19 Vaccine, JNJ-78436735, ZF2001, CVnCoV, INO-4800, VIR-7831, Covid-19 adenovirus bases vaccine, UB-612, or CoVaxin.

48-63. (canceled)

64. A nucleotide sequence encoding a SARS-CoV-2 vaccine of claim 1.

65. The nucleotide sequence of claim 64, wherein the nucleotide sequence comprises DNA.

66. The nucleotide sequence of claim 64, wherein the nucleotide sequence comprises RNA.

67. The nucleotide sequence of claim 64, wherein the nucleotide sequence comprises messenger RNA (mRNA).


SEQUENCE LISTING (293 sequences)

SEQ ID NO: 1 - protein, 1193 aa, Synthetic Construct
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 2 - protein, 1532 aa, Synthetic Construct
METDTLLLWVLLLWVPGSTGDYKDEVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 3 - protein, 1507 aa, Synthetic Construct
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 4 - protein, 1476 aa, Synthetic Construct
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGGGSGGGSGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 5 - protein, 206 aa, Synthetic Construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNL

SEQ ID NO: 6 - protein, 197 aa, Synthetic Construct
CEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGL

SEQ ID NO: 7 - protein, 471 aa, Synthetic Construct
METDTLLLWVLLLWVPGSTGDYKDERFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 8 - protein, 446 aa, Synthetic Construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 9 - protein, 415 aa, Synthetic Construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLGGGSGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGS

SEQ ID NO: 10 - protein, 20 aa, Synthetic Construct
GAISSVLNDILSRLDKVEAE

SEQ ID NO: 11 - protein, 5 aa, Synthetic Construct
GPGPG

SEQ ID NO: 12-15 - skipped (4 entries)

SEQ ID NO: 16 - protein, 100 aa, Synthetic Construct
MGKSPTEVLLELIAEASGTTREEVKEKFLKELRKGKSPTEVLLELIAEASGTTKEEVKEKFLKELSFGKSPTEVLLELIAEASGTTKEEVKKKFWKELSL

SEQ ID NO: 17 - protein, 13 aa, Synthetic Construct
PTEVLLELIAEAS

SEQ ID NO: 18 - protein, 14 aa, Synthetic Construct
REEVKEKFLKELRK

SEQ ID NO: 19 - protein, 14 aa, Synthetic Construct
KEEVKEKFLKELSF

SEQ ID NO: 20 - protein, 14 aa, Synthetic Construct
KEEVKKKFWKELSL

SEQ ID NO: 21 - protein, 33 aa, Synthetic Construct
GKSPTEVLLELIAEASGTTREEVKEKFLKELRK

SEQ ID NO: 22 - protein, 33 aa, Synthetic Construct
GKSPTEVLLELIAEASGTTKEEVKEKFLKELSF

SEQ ID NO: 23 - protein, 33 aa, Synthetic Construct
GKSPTEVLLELIAEASGTTKEEVKKKFWKELSL

SEQ ID NO: 24 - protein, 199 aa, Synthetic Construct
MGDKTAIAQILAIKASAKGDETELERALRYAVKVGDKTAIAQILAIKASAKGDETELEQALRYAKFVGDKTAIAQILAIKASAKGDELELTRALAYAKKVGDKTAIAQILAIKASAKGDETELERALRYAKLVGDKTAIAQILAIKASAKGDETELERALRYAKYVGDKTAIAQILAIKASAKGDEPELEYALAYAKKV

SEQ ID NO: 25 - protein, 14 aa, Synthetic Construct
TAIAQILAIKASAK

SEQ ID NO: 26 - protein, 13 aa, Synthetic Construct
TELERALRYAVKV

SEQ ID NO: 27 - protein, 13 aa, Synthetic Construct
TELEQALRYAKFV

SEQ ID NO: 28 - protein, 13 aa, Synthetic Construct
LELTRALAYAKKV

SEQ ID NO: 29 - protein, 13 aa, Synthetic Construct
TELERALRYAKLV

SEQ ID NO: 30 - protein, 13 aa, Synthetic Construct
TELERALRYAKYV

SEQ ID NO: 31 - protein, 13 aa, Synthetic Construct
PELEYALAYAKKV

SEQ ID NO: 32 - protein, 33 aa, Synthetic Construct
GDKTAIAQILAIKASAKGDETELERALRYAVKV

SEQ ID NO: 33 - protein, 33 aa, Synthetic Construct
GDKTAIAQILAIKASAKGDETELEQALRYAKFV

SEQ ID NO: 34 - protein, 33 aa, Synthetic Construct
GDKTAIAQILAIKASAKGDELELTRALAYAKKV

SEQ ID NO: 35 - protein, 33 aa, Synthetic Construct
GDKTAIAQILAIKASAKGDETELERALRYAKLV

SEQ ID NO: 36 - protein, 33 aa, Synthetic Construct
GDKTAIAQILAIKASAKGDETELERALRYAKYV

SEQ ID NO: 37 - protein, 33 aa, Synthetic Construct
GDKTAIAQILAIKASAKGDEPELEYALAYAKKV

SEQ ID NO: 38 - protein, 221 aa, Synthetic Construct
LVSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLGVSLEQALKILKVAAELGTTVEEAVKRALKLKTKLGVSLEQALKILEVAAELGTTVEEAVKRAMKLKTKLGVSLEQALKILEVAAKLGTTVEEAVKRALKLKTKLGGWLEHHHHHH

SEQ ID NO: 39 - protein, 14 aa, Synthetic Construct
LEQALKILKVAAEL

SEQ ID NO: 40 - protein, 15 aa, Synthetic Construct
VEEAVKRALKLKTKL

SEQ ID NO: 41 - protein, 14 aa, Synthetic Construct
LEQALKILEVAAEL

SEQ ID NO: 42 - protein, 14 aa, Synthetic Construct
LEQALKILEVAAKL

SEQ ID NO: 43 - protein, 15 aa, Synthetic Construct
VEEAVKRAMKLKTKL

SEQ ID NO: 44 - protein, 35 aa, Synthetic Construct
LVSLEQALKILKVAAELGTTVEEAVKRALKLKTKL

SEQ ID NO: 45 - protein, 35 aa, Synthetic Construct
GVSLEQALKILKVAAELGTTVEEAVKRALKLKTKL

SEQ ID NO: 46 - protein, 35 aa, Synthetic Construct
GVSLEQALKILEVAAELGTTVEEAVKRALKLKTKL

SEQ ID NO: 47 - protein, 35 aa, Synthetic Construct
GVSLEQALKILEVAAKLGTTVEEAVKRALKLKTKL

SEQ ID NO: 48 - protein, 35 aa, Synthetic Construct
GVSLEQALKILEVAAELGTTVEEAVKRAMKLKTKL

SEQ ID NO: 49 - protein, 286 aa, Synthetic Construct
GSSMASGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLG

SEQ ID NO: 50 - protein, 379 aa, Synthetic Construct
GSSMASGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLG

SEQ ID NO: 51 - protein, 100 aa, Synthetic Construct
GSSMASGISVEELLKLAKAAYYSGTTVEEAYKLALKLGISVEELLKLAEAAYYSGTTVEEAYKLALKLGISVEELLKLAKAAYYSGTTVEEAYKLALKLG

SEQ ID NO: 52 - protein, 14 aa, Synthetic Construct
VEELLKLAKAAYYS

SEQ ID NO: 53 - protein, 11 aa, Synthetic Construct
VEEAYKLALKL

SEQ ID NO: 54 - protein, 14 aa, Synthetic Construct
VEELLKLAEAAYYS

SEQ ID NO: 55 - protein, 31 aa, Synthetic Construct
GISVEELLKLAKAAYYSGTTVEEAYKLALKL

SEQ ID NO: 56 - protein, 31 aa, Synthetic Construct
GISVEELLKLAEAAYYSGTTVEEAYKLALKL

SEQ ID NO: 57 - protein, 795 aa, Synthetic Construct
GLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGLGLNPEAIKAAAELGKAGISSEEILELLRAAHELGLNPEAIKAAAELGKAGISSEEILELLRAAHELGLNPEAIKAAAELGKAGISSEEILELLRAAHALGLNPEAIKAAAELGKAGISSAEILELLMAAHELGLNPEAIKAAAELGKAGISSEEILELLRAAHELGLNPEAIKAAAELGKAGISSEEILELLRAAHGGW

SEQ ID NO: 58 - protein, 13 aa, Synthetic Construct
PEAIKAAAELGKA

SEQ ID NO: 59 - protein, 14 aa, Synthetic Construct
SEEILELLRAAHEL

SEQ ID NO: 60 - protein, 14 aa, Synthetic Construct
SEEILELLRAAHAL

SEQ ID NO: 61 - protein, 14 aa, Synthetic Construct
SAEILELLMAAHEL

SEQ ID NO: 62 - protein, 14 aa, Synthetic Construct
SEEILELLRAAHGL

SEQ ID NO: 63 - protein, 14 aa, Synthetic Construct
SEEILELLRAAHGG

SEQ ID NO: 64 - protein, 33 aa, Synthetic Construct
GLNPEAIKAAAELGKAGISSEEILELLRAAHEL

SEQ ID NO: 65 - protein, 33 aa, Synthetic Construct
GLNPEAIKAAAELGKAGISSEEILELLRAAHAL

SEQ ID NO: 66 - protein, 33 aa, Synthetic Construct
GLNPEAIKAAAELGKAGISSAEILELLMAAHEL

SEQ ID NO: 67 - protein, 33 aa, Synthetic Construct
GLNPEAIKAAAELGKAGISSEEILELLRAAHGL

SEQ ID NO: 68 - protein, 33 aa, Synthetic Construct
GLNPEAIKAAAELGKAGISSEEILELLRAAHGG

SEQ ID NO: 69 - protein, 271 aa, Synthetic Construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHVLGLDPEAIKAAAELGKAGISSEEILELLVAAHLLGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSIEILELLRAAHELGGW

SEQ ID NO: 70 - protein, 203 aa, Synthetic Construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELG

SEQ ID NO: 71 - protein, 137 aa, Synthetic Construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELG

SEQ ID NO: 72 - protein, 104 aa, Synthetic Construct
GSSMGLDPEAIKAAAELGKAGISSEEILELLRAAHELGLDPEAIKAAAELGKAGISSEEILELLRAAHELGLDPEAIKAAAELGKAGISSEEILELLRAAHELG

SEQ ID NO: 73 - protein, 14 aa, Synthetic Construct
SEEILELLRAAHVL

SEQ ID NO: 74 - protein, 14 aa, Synthetic Construct
SIEILELLRAAHEL

SEQ ID NO: 75 - protein, 33 aa, Synthetic Construct
GLDPEAIKAAAELGKAGISSEEILELLRAAHEL

SEQ ID NO: 76 - protein, 33 aa, Synthetic Construct
GLDPEAIKAAAELGKAGISSEEILELLRAAHVL

SEQ ID NO: 77 - protein, 33 aa, Synthetic Construct
GLDPEAIKAAAELGKAGISSEEILELLVAAHLL

SEQ ID NO: 78 - protein, 33 aa, Synthetic Construct
GLDPEAIKAAAELGKAGISSIEILELLRAAHEL

SEQ ID NO: 79 - protein, 514 aa, Synthetic Construct
GNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEG

SEQ ID NO: 80 - protein, 24 aa, Synthetic Construct
ELALKALQILVNAAYVLAEIARDR

SEQ ID NO: 81 - protein, 27 aa, Synthetic Construct
ELLEKAARLAEEAARQAEEIARQARKE

SEQ ID NO: 82 - protein, 57 aa, Synthetic Construct
GNLELALKALQILVNAAYVLAEIARDRGNEELLEKAARLAEEAARQAEEIARQARKE

SEQ ID NO: 83 - protein, 93 aa, Synthetic Construct
GISVEELLKLAKAAYYSGTTVEEAYKLALKLGISVEELLKLAEAAYYSGTTVEEAYKLALKLGISVEELLKLAKAAYYSGTTVEEAYKLALKL

SEQ ID NO: 84 - protein, 14 aa, Synthetic Construct
SEEILELLVAAHLL

SEQ ID NO: 85 - protein, 14 aa, Synthetic Construct
SEEIKELLRAAHEL

SEQ ID NO: 86 - protein, 14 aa, Synthetic Construct
SEEILELLRAAHEK

SEQ ID NO: 87 - protein, 13 aa, Synthetic Construct
PEAEKAAAELGKA

SEQ ID NO: 88 - protein, 13 aa, Synthetic Construct
PEAIKKAAELGKA

SEQ ID NO: 89 - protein, 13 aa, Synthetic Construct
PECIKAAAELGKA

SEQ ID NO: 90 - protein, 13 aa, Synthetic Construct
PELEYALAYAKKV

SEQ ID NO: 91 - protein, 25 aa, Synthetic Construct
SELAARILIILFQQLVELARLAIES

SEQ ID NO: 92 - protein, 28 aa, Synthetic Construct
EELLRRVSEWLEEVIKDMRRVVEQALRE

SEQ ID NO: 93 - protein, 27 aa, Synthetic Construct
ELLRRVSEWLEEVIKDMRRVVEQALRE

SEQ ID NO: 94 - protein, 25 aa, Synthetic Construct
LELALKALQILVNAAYVLAEIARDR

SEQ ID NO: 95 - protein, 13 aa, Synthetic Construct
CEAIKAAAELGKA

SEQ ID NO: 96 - protein, 25 aa, Synthetic Construct
SELAARCLIILFQQLVELARLAIES

SEQ ID NO: 97 - protein, 13 aa, Synthetic Construct
CEAEKAAAELGKA

SEQ ID NO: 98 - protein, 13 aa, Synthetic Construct
CEAIKKAAELGKA

SEQ ID NO: 99 - protein, 13 aa, Synthetic Construct
CECIKAAAELGKA

SEQ ID NO: 100 - protein, 13 aa, Synthetic Construct
CTEVLLELIAEAS

SEQ ID NO: 101 - protein, 13 aa, Synthetic Construct
CELEYALAYAKKV

SEQ ID NO: 102 - skipped

SEQ ID NO: 103 - protein, 25 aa, Synthetic Construct
SELACRILIILFQQLVELARLAIES

SEQ ID NO: 104 - protein, 13 aa, Synthetic Construct
PECEKAAAELGKA

SEQ ID NO: 105 - protein, 13 aa, Synthetic Construct
PECIKKAAELGKA

SEQ ID NO: 106 - skipped

SEQ ID NO: 107 - protein, 16 aa, Synthetic Construct
GLDPEAIKAAAELGKA

SEQ ID NO: 108 - protein, 27 aa, Synthetic Construct
GNSELAARILIILFQQLVELARLAIES

SEQ ID NO: 109 - protein, 16 aa, Synthetic Construct
GDEPELEYALAYAKKV

SEQ ID NO: 110 - protein, 16 aa, Synthetic Construct
GLNPEAIKAAAELGKA

SEQ ID NO: 111 - protein, 16 aa, Synthetic Construct
GLDCEAIKAAAELGKA

SEQ ID NO: 112 - protein, 27 aa, Synthetic Construct
GNSELAARCLIILFQQLVELARLAIES

SEQ ID NO: 113 - protein, 16 aa, Synthetic Construct
GDECELEYALAYAKKV

SEQ ID NO: 114 - protein, 16 aa, Synthetic Construct
GLNCEAIKAAAELGKA

SEQ ID NO: 115 - protein, 16 aa, Synthetic Construct
GLNCEAEKAAAELGKA

SEQ ID NO: 116 - protein, 16 aa, Synthetic Construct
GLDCEAEKAAAELGKA

SEQ ID NO: 117 - protein, 16 aa, Synthetic Construct
GLDCEAIKKAAELGKA

SEQ ID NO: 118 - protein, 16 aa, Synthetic Construct
GLNCEAIKKAAELGKA

SEQ ID NO: 119 - protein, 16 aa, Synthetic Construct
GLDPECIKAAAELGKA

SEQ ID NO: 120 - protein, 27 aa, Synthetic Construct
GNSELACRILIILFQQLVELARLAIES

SEQ ID NO: 121 - protein, 16 aa, Synthetic Construct
GLNPECIKAAAELGKA

SEQ ID NO: 122 - protein, 27 aa, Synthetic Construct
GLNELCARILIILFQQLVELARLAIES

SEQ ID NO: 123 - protein, 27 aa, Synthetic Construct
GLNELCLKALQILVNAAYVLAEIARDR

SEQ ID NO: 124 - protein, 19 aa, Synthetic Construct
GLDPEAIKAAAELGKAGIS

SEQ ID NO: 125 - protein, 29 aa, Synthetic Construct
GNSELAARILIILFQQLVELARLAIESGD

SEQ ID NO: 126 - protein, 19 aa, Synthetic Construct
GKSPTEVLLELIAEASGTT

SEQ ID NO: 127 - protein, 19 aa, Synthetic Construct
GLNPEAIKAAAELGKAGIS

SEQ ID NO: 128 - protein, 19 aa, Synthetic Construct
GLDCEAIKAAAELGKAGIS

SEQ ID NO: 129 - protein, 29 aa, Synthetic Construct
GNSELAARCLIILFQQLVELARLAIESGD

SEQ ID NO: 130 - protein, 19 aa, Synthetic Construct
GKSCTEVLLELIAEASGTT

SEQ ID NO: 131 - protein, 19 aa, Synthetic Construct
GLNCEAIKAAAELGKAGIS

SEQ ID NO: 132 - protein, 19 aa, Synthetic Construct
GLNCEAEKAAAELGKAGTT

SEQ ID NO: 133 - protein, 19 aa, Synthetic Construct
GLDCEAEKAAAELGKAGIS

SEQ ID NO: 134 - protein, 19 aa, Synthetic Construct
GLDCEAIKKAAELGKAGTT

SEQ ID NO: 135 - protein, 19 aa, Synthetic Construct
GLDCEAIKKAAELGKAGIS

SEQ ID NO: 136 - protein, 19 aa, Synthetic Construct
GLDPECIKAAAELGKAGIS

SEQ ID NO: 137 - protein, 29 aa, Synthetic Construct
GNSELACRILIILFQQLVELARLAIESGD

SEQ ID NO: 138 - protein, 19 aa, Synthetic Construct
GLNPECIKAAAELGKAGIS

SEQ ID NO: 139 - protein, 57 aa, Synthetic Construct
GNSELAARILIILFQQLVELARLAIESGDEELLRRVSEWLEEVIKDMRRVVEQALRE

SEQ ID NO: 140 - protein, 33 aa, Synthetic Construct
GLDCEAIKAAAELGKAGISSEEILELLRAAHEL

SEQ ID NO: 141 - protein, 57 aa, Synthetic Construct
GNSELAARCLIILFQQLVELARLAIESGDEELLRRVSEWLEEVIKDMRRVVEQALRE

SEQ ID NO: 142 - protein, 33 aa, Synthetic Construct
GKSCTEVLLELIAEASGTTREEVKEKFLKELRK

SEQ ID NO: 143 - protein, 33 aa, Synthetic Construct
GKSCTEVLLELIAEASGTTKEEVKEKFLKELSF

SEQ ID NO: 144 - protein, 33 aa, Synthetic Construct
GKSCTEVLLELIAEASGTTKEEVKKKFWKELSL

SEQ ID NO: 145 - protein, 33 aa, Synthetic Construct
GLDCEAIKAAAELGKAGISSEEILELLRAAHVL

SEQ ID NO: 146 - protein, 33 aa, Synthetic Construct
GLDCEAIKAAAELGKAGISSEEILELLVAAHLL

SEQ ID NO: 147 - protein, 33 aa, Synthetic Construct
GLDCEAIKAAAELGKAGISSIEILELLRAAHEL

SEQ ID NO: 148 - protein, 33 aa, Synthetic Construct
GLNCEAIKAAAELGKAGISSEEILELLRAAHEL

SEQ ID NO: 149 - protein, 33 aa, Synthetic Construct
GLNCEAIKAAAELGKAGISSEEILELLRAAHAL

SEQ ID NO: 150 - protein, 33 aa, Synthetic Construct
GLNCEAIKAAAELGKAGISSAEILELLMAAHEL

SEQ ID NO: 151 - protein, 33 aa, Synthetic Construct
GLNCEAIKAAAELGKAGISSEEILELLRAAHGL

SEQ ID NO: 152 - protein, 33 aa, Synthetic Construct
GLNCEAIKAAAELGKAGISSEEILELLRAAHGG

SEQ ID NO: 153 - protein, 33 aa, Synthetic Construct
GLDPECIKAAAELGKAGISSEEILELLRAAHEL

SEQ ID NO: 154 - protein, 57 aa, Synthetic Construct
GNSELACRILIILFQQLVELARLAIESGDEELLRRVSEWLEEVIKDMRRVVEQALRE

SEQ ID NO: 155 - protein, 33 aa, Synthetic Construct
GLDPECIKAAAELGKAGISSEEILELLRAAHVL

SEQ ID NO: 156 - protein, 33 aa, Synthetic Construct
GLDPECIKAAAELGKAGISSEEILELLVAAHLL

SEQ ID NO: 157 - protein, 33 aa, Synthetic Construct
GLDPECIKAAAELGKAGISSIEILELLRAAHEL

SEQ ID NO: 158 - protein, 33 aa, Synthetic Construct
GLNPECIKAAAELGKAGISSEEILELLRAAHEL

SEQ ID NO: 159 - protein, 33 aa, Synthetic Construct
GLNPECIKAAAELGKAGISSEEILELLRAAHAL

SEQ ID NO: 160 - protein, 33 aa, Synthetic Construct
GLNPECIKAAAELGKAGISSAEILELLMAAHEL

SEQ ID NO: 161 - protein, 33 aa, Synthetic Construct
GLNPECIKAAAELGKAGISSEEILELLRAAHGL

SEQ ID NO: 162 - protein, 33 aa, Synthetic Construct
GLNPECIKAAAELGKAGISSEEILELLRAAHGG

SEQ ID NO: 163 - DNA, 2454 bp, Synthetic Construct
  source 1..2454: mol_type = other DNA
atggctagcagccatcatcatcatcatcatagcagcggcctggtgccgcgcggcagctccatgggcctggacccggaagcgatcaaagcggctgcggaactgggcaaagctggtatctcgtcagaagaaatcctggaactgctgcgtgcggcgcatgaactgggcctggatccggaagccattaaagccgcggcggaactgggcaaagcgggtatcagcagcgaagaaatcctggaactgctgcgcgcggcccatgaactgggtctggatccggaagccatcaaagccgcggcggaactgggcaaagcgggcatcagcagcgaagaaatcctggaactgctgcgcgccgcgcatgaactgggtctggatccggaagcgattaaagccgccgccgaactgggtaaagcgggtattagcagcgaagaaatcctggaactgctgcgtgcggcgcatgaactgggcctggatccggaagccatcaaagccgccgccgaactgggtaaagccggtatcagcagcgaagaaattctggaactgctgcgtgccgcccatgaactgggcctggatccggaagcgattaaagccgccgcggaactgggtaaagccggtattagcagcgaagaaatcctggaactgctgcgtgccgcgcatgaactgggtctggatccggaagccatcaaagcggcggccgaactgggtaaagccggcatcagcagcgaagaaatcctggaactgctgcgtgccgcccatgaactgggcctggatccggaagcgatcaaagccgccgcggaactgggtaaagccggcattagcagcgaagaaatcctggaactgctgcgtgccgcgcatgaactgggtctggatccggaagcgattaaagcggcggccgaactgggcaaagccggtattagcagcgaagaaatcctggaactgctgcgtgcggcccatgaactgggcctggatccggaagcgatcaaagcggcggccgaactgggtaaagcgggtatcagcagcgaagaaatcctggaactgctgcgtgccgcccatgaactgggtctggatccggaagccattaaagcggcggcggaactgggtaaagcgggcatcagcagcgaagaaattctggaactgctgcgcgcggcgcatgaactgggtctggatccggaagccattaaagccgccgcggaactgggcaaagcgggtattagcagcgaagaaattctggaactgctgcgcgcggcgcatgaactgggcctggatccggaagcgattaaagccgccgccgaactgggcaaagccggcatcagcagcgaagaaattctggaactgctgcgcgccgcccatgaactgggcctggatccggaagcgatcaaagccgccgcggaactgggcaaagcgggcatcagcagcgaagaaatcctggaactgctgcgtgcggcgcatgaactgggtctggatccggaagccattaaagccgcggcggaactgggcaaagccggcattagcagcgaagaaattctggaactgctgcgtgcggcgcatgaactgggtctggatccggaagccatcaaagccgcggcggaactgggtaaagcgggtattagcagcgaagaaattctggaactgctgcgcgcggcccatgaactgggcctggatccggaagcgattaaagcggcggccgaactgggcaaagcgggcattagcagcgaagaaatcctggaactgctgcgcgcggcgcatgaactgggtctggatccggaagccattaaagccgccgccgaactgggcaaagccggtatcagcagcgaagaaattctggaactgctgcgcgcggcccatgaactgggtctggatccggaagccatcaaagccgccgcggaactgggtaaagccggtatcagcagcgaagaaatcctggaactgctgcgcgccgcccatgaactgggcctggatccggaagcgatcaaagccgcggcggaactgggtaaagcgggcattagcagcgaagaaattctggaactgctgcgtgcggcccatgaactgggtctggatccggaagcgatcaaagccgcggccgaactgggtaaagcgggcatcagcagcgaagaaattctggaactgctgcgtgccgcgcatgaactgggcctggatccggaagcgattaaagccgcggccgaactgggcaaagcgggcattagcagcgaagaaattctggaactgctgcgcgcggcgcatgaactgggcctggatccggaagcgattaaagcggcggccgaactgggcaaagcgggtatcagcagcgaagaaatcctggaactgctgcgcgccgcgcatgaactgggcctggatccggaagcgattaaagccgcggccgaactgggcaaagcgggtatctcgtctgaagaaatcctggaactgctgcgtgctgcccatgaactgggtggttggtaataa

SEQ ID NO: 164 - protein, 816 aa, Synthetic Construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 165 - DNA, 372 bp, Synthetic Construct
  source 1..372: mol_type = other DNA
atggctagcagccatcatcatcatcatcatagcagcggcctggtgccgcgcggcagctccatgggcctggatccggaagctattaaagcggcggcagaactgggcaaagccggtattagctctgaagaaatcctggaactgctgcgtgctgcgcatgaactgggtctggacccggaagcaatcaaagccgcagctgaactgggcaaagcgggtattagttccgaagaaattctggaactgctgcgtgccgcacatgaactgggtctggatccggaagccattaaagcagctgcggaactgggtaaagccggtattagcagcgaggagattctggaactgctgcgcgccgcacatgaactgggcggttggtaa

SEQ ID NO: 166 - protein, 123 aa, Synthetic Construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 167 - DNA, 471 bp, Synthetic Construct
  source 1..471: mol_type = other DNA
atggctagcagccatcatcatcatcatcatagcagcggcctggtgccgcgcggcagctccatgggcctggatccggaagcgattaaagcggcggcagaactgggcaaagccggtattagctctgaagaaatcctggaactgctgcgtgctgcgcatgaactgggtctggacccggaagcaatcaaagccgcagctgaactgggcaaagctggtattagttccgaagaaattctggaactgctgcgtgccgcacatgaactgggtctggatccggaagccatcaaagcagctgcggaactgggtaaagcgggtatttcatcggaggagatcctggaactgctgcgcgcggcccacgaactgggcctggatccggaagctatcaaagctgcggccgaactgggtaaagctggtattagcagcgaggagattctggaactgctgcgcgcagctcacgaactgggcggttggtaa

SEQ ID NO: 168 - protein, 156 aa, Synthetic Construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 169 - DNA, 669 bp, Synthetic Construct
  source 1..669: mol_type = other DNA
atggctagcagccatcatcatcatcatcatagcagcggcctggtgccgcgcggcagctccatgggcctggatccggaagcaattaaagcggcggcagaactgggcaaagctggtattagctctgaagaaatcctggaactgctgcgtgctgcgcatgaactgggtctggacccggaagcaatcaaagccgcagctgaactgggcaaagcgggtattagttccgaagaaattctggaactgctgcgtgccgcacatgaactgggtctggatccggaagccattaaagcagctgcggaactgggcaaagcaggtatttcatcggaggagatcctggaactgctgcgcgcggcccacgaactgggcctggacccggaagcgatcaaagctgcggccgaactgggcaaagccggcatctctagcgaggagattctggaactgctgcgcgcagctcacgaactgggcctggatccggaagcgatcaaggcggcggcagagctgggcaaagccggtattagttcagaggagatactggaactgctgcgcgccgcccacgagttaggtctggatccggaagcgattaaggccgcagctgagctgggtaaagccggtattagctccgaggagatattagaactgctgcgtgccgcgcatgaactgggcggttggtaa

SEQ ID NO: 170 - protein, 222 aa, Synthetic Construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 171 - protein, 261 aa, Synthetic Construct
METDTLLLWVLLLWVPGSTGDYKDEGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 172 - DNA, 867 bp, Synthetic Construct
  source 1..867: mol_type = other DNA
atggctagcagccatcatcatcatcatcatagcagcggcctggtgccgcgcggcagctccatgggcctggatccggaagcgattaaagcggcggcagaactgggcaaagccggtattagctctgaagaaatcctggaactgctgcgtgctgcgcatgaactgggtctggacccggaagcaatcaaagccgcagctgaactgggcaaagctggtattagttccgaagaaattctggaactgctgcgtgccgcacatgaactgggtctggatccggaagcaattaaagcagctgcggaactgggcaaagcgggtatttcatcggaggagatcctggaactgctgcgcgccgcacatgtgctgggcctggatccggaagccatcaaagctgcggccgaactgggcaaagcaggcatctctagcgaggagattctggaactgctggttgcagctcacctgctgggtttagaccctgaagccatcaaggcggcggcagagctgggtaaagcgggcatcagttccgaggagatactggaactgctgcgcgcggcccacgaactgggcctggacccggaagcgattaaggccgcagctgagctgggtaaagcgggtatcagctcggaggagatattagaactgctgcgcgccgcccatgagttaggcctggacccggaagccatcaaggcagctgcggagctgggtaaagctggcatctctagcgaggaaattttagagctgctgcgtgcggcccacgagttaggtctggacccggaagccattaaggctgcggccgagctgggcaaagcgggtatctcatccattgagatattggagctgctgcgtgcagctcacgaactgggcggttggtaa

SEQ ID NO: 173 - protein, 288 aa, Synthetic Construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHVLGLDPEAIKAAAELGKAGISSEEILELLVAAHLLGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSIEILELLRAAHELGGW

SEQ ID NO: 174 - protein, 327 aa, Synthetic Construct
METDTLLLWVLLLWVPGSTGDYKDEGGGSCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGLGGGSENLYFQGSHHHHHHGSGLNDIFEAQKIEWHE

SEQ ID NO: 175 - DNA, 1266 bp, Synthetic Construct
  source 1..1266: mol_type = other DNA
atggctagcagccatcatcatcatcatcatagcagcggcctggtgccgcgcggcagctccatgggcctggacccggaggcgatcaaggctgcggcggaactgggtaaagcgggcatcagcagcgaggaaattctggagctgctgcgtgcggcgcatgagctgggtctggacccggaagcgattaaggctgcggcggagctgggcaaggcgggtatcagcagcgaagaaattctggaactgctgcgcgcggcgcatgagctgggcctggacccggaagcgatcaaagctgcggcggagctgggtaaggctggtatcagcagcgaggagattctggaattattacgcgcggcgcatgaactgggtctggacccggaggcgattaaagctgcggcggaactgggcaaggcgggcatcagcagcgaagagattctggaattacttcgtgctgcgcatgaactgggcctggacccggaggctattaaggctgcggcggaattaggcaaagctggcatcagcagcgaagaaattctggaattactgcgtgccgctcatgaattaggtctggacccggaagctatcaaagctgcggcggaattaggtaaggccggtatcagcagcgaggaaattctggaattgttacgagctgctcatgaattaggcctggacccggaggccattaaggctgcggcggagttaggcaaagcgggtatcagcagcgaggaaattctggaacttttacgagctgctcatgagttaggtctggacccggaggctatcaaggctgcggcggagttgggcaaagccggcatcagcagcgaggagattctggaacttttacgggctgctcatgaacttggcctggacccggaggcaattaaggctgcggcggagctaggcaaggctggtatcagcagcgaggaaattctggaactactgcgtgccgcccatgaattgggtctggacccggaagcaatcaaggctgcggcggagcttggcaaggccggtatcagcagcgaggaaattctggaactgttacgagctgcccatgaacttggtctggacccggaagccataaaagctgcggcggagttaggtaaggcaggtatcagcagcgaggaaattctggaactcctgcgtgccgcacatgaactcggtctggacccggaggccattaaagctgcggcggagttgggtaaggcgggtatcagcagcgaggaaattctggagttactgcgtgccgcgcatgagctgggtggctggtaataa

SEQ ID NO: 176 - protein, 420 aa, Synthetic Construct
MASSHHHHHHSSGLVPRGSSMGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGGW

SEQ ID NO: 177 - DNA, 1266 bp, Synthetic Construct
  source 1..1266: mol_type = other DNA
atggctagcagccatcatcatcatcatcatagcagcggcctggtgccgcgcggcagctccatgggcctggactgcgaggcgatcaaggctgcggcggaactgggtaaagcgggcatcagcagcgaggaaattctggagctgctgcgtgcggcgcatgagctgggtctggacccggaagcgattaaggctgcggcggagctgggcaaggcgggtatcagcagcgaagaaattctggaactgctgcgcgcggcgcatgagctgggcctggacccggaagcgatcaaagctgcggcggagctgggtaaggctggtatcagcagcgaggagattctggaattattacgcgcggcgcatgaactgggtctggacccggaggcgattaaagctgcggcggaactgggcaaggcgggcatcagcagcgaagagattctggaattacttcgtgctgcgcatgaactgggcctggacccggaggctattaaggctgcggcggaattaggcaaagctggcatcagcagcgaagaaattctggaattactgcgtgccgctcatgaattaggtctggacccggaagctatcaaagctgcggcggaattaggtaaggccggtatcagcagcgaggaaattctggaattgttacgagctgctcatgaattaggcctggacccggaggccattaaggctgcggcggagttaggcaaagcgggtatcagcagcgaggaaattctggaacttttacgagctgctcatgagttaggtctggacccggaggctatcaaggctgcggcggagttgggcaaagccggcatcagcagcgaggagattctggaacttttacgggctgctcatgaacttggcctggacccggaggcaattaaggctgcggcggagctaggcaaggctggtatcagcagcgaggaaattctggaactactgcgtgccgcccatgaattgggtctggacccggaagcaatcaaggctgcggcggagcttggcaaggccggtatcagcagcgaggaaattctggaactgttacgagctgcccatgaacttggtctggacccggaagccataaaagctgcggcggagttaggtaaggcaggtatcagcagcgaggaaattctggaactcctgcgtgccgcacatgaactcggtctggacccggaatgcataaaggctgcggcggaattgggcaaagctggtatcagcagcgaggaaattctggagttactgcgtgccgcgcatgagctgggtggctggtaataa

SEQ ID NO: 178 - protein, 420 aa, Synthetic Construct
MASSHHHHHHSSGLVPRGSSMGLDCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGGW

SEQ ID NO: 179 - DNA, 1266 bp, Synthetic Construct
  source 1..1266: mol_type = other DNA
atggctagcagccatcatcatcatcatcatagcagcggcctggtgccgcgcggcagctccatgggcctggacccggaggcggaaaaggctgcggcggagctgggtaaagcgggcatcagcagcgaggaaattaaagaactgctgcgtgcggcgcatgagctgggtctggacccggaagcgatcaaagctgcggcggaactgggcaaggcgggtatcagcagcgaagaaattctggaactgctgcgcgcggcgcatgagctgggcctggacccggaagcgattaaagctgcggcggagctgggcaaggcgggcatcagcagcgaagagattctggaattattacgcgcggcgcatgaactgggtctggacccggaggcgatcaaggctgcggcggaactgggtaaggctggtatcagcagcgaggagattctggaattacttcgtgctgcgcatgaactgggcctggacccggaggcgattaaggctgcggcggagttaggcaaagctggcatcagcagcgaagaaattctggaattactgcgtgccgctcatgaattaggtctggacccggaagctatcaaggctgcggcggagttgggcaaagccggcatcagcagcgaggagattctggaacttttacgagctgctcatgaattaggcctggacccggaagctattaaagctgcggcggaattaggtaaggccggtatcagcagcgaggaaattctggaattgttacgagctgctcatgagttaggtctggacccggaggctatcaaagctgcggcggagttaggtaaggcaggtatcagcagcgaggaaattctggaacttttacgggctgctcatgaacttggcctggacccggaagccatcaaagctgcggcggaattagggaaagctggaatcagcagcgaagagattctggaacttcttcgtgccgctcatgaacttggtctggacccggaggccattaaagctgcggcggaacttggtaaggcgggtatcagcagcgaggaaattctggaactactgcgtgccgcccatgaattgggtctggacccggaagcaatcaaggctgcggcggagcttggcaaagcaggtatcagcagcgaggaaattctggagttattgcgtgccgctcatgaactcggtctggacccggaagccatcaagaaagcggcggaactgggcaaagcaggcatcagcagcgaagagattctggagctgctgcgtgcggcgcacgaaaaaggtggctggtaataa

SEQ ID NO: 180 - protein, 420 aa, Synthetic Construct
MASSHHHHHHSSGLVPRGSSMGLDPEAEKAAAELGKAGISSEEIKELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKKAAELGKAGISSEEILELLRAAHEKGGW

SEQ ID NO: 181 - DNA, 669 bp, Synthetic Construct
  source 1..669: mol_type = other DNA
atggctagcagccatcatcatcatcatcatagcagcggcctggtgccgcgcggcagctccatgggcctggactgcgaggccattaaggctgcggcggagttaggcaaagcgggtatcagcagcgaggaaattctggaacttttacgagctgctcatgagttaggtctggacccggaggctatcaaggctgcggcggagttgggcaaagccggcatcagcagcgaggagattctggaacttttacgggctgctcatgaacttggcctggacccggaggcaattaaggctgcggcggagctaggcaaggctggtatcagcagcgaggaaattctggaactactgcgtgccgcccatgaattgggtctggacccggaagcaatcaaggctgcggcggagcttggcaaggccggtatcagcagcgaggaaattctggaactgttacgagctgcccatgaacttggtctggacccggaagccataaaagctgcggcggagttaggtaaggcaggtatcagcagcgaggaaattctggaactcctgcgtgccgcacatgaactcggtctggacccggagtgcattaaagctgcggcggagttgggtaaggcgggtatcagcagcgaggaaattctggagttactgcgtgccgcgcatgagctgggtggctggtaa

SEQ ID NO: 182 - protein, 222 aa, Synthetic Construct
MASSHHHHHHSSGLVPRGSSMGLDCEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGGW

SEQ ID NO: 183 - protein, 172 aa, Synthetic Construct
GNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEGNLELALKALQILVNAAYVLAEIARDRGNEELLEKAARLAEEAARQAEEIARQARKEG

SEQ ID NO: 184 - protein, 342 aa, Synthetic Construct
GNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALRE

SEQ ID NO: 185 - protein, 457 aa, Synthetic Construct
GNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREG

SEQ ID NO: 186 - protein, 457 aa, Synthetic Construct
GNSELAARCLIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELAARILIILFQQLVELARLAIESGDEELLRRVSEWLEEVIKDMRRVVEQALREGNSELACRILIILFQQLVELARLAIESGDEELLRRVSEWLEEVIKDMRRVVEQALREG

SEQ ID NO: 187 - protein, 99 aa, Synthetic Construct
GKSPTEALLKLIAEAKGITETEAKEEAEKALKEGKSPTEALLKLIAEAKGITETEAKEEAEKALKEGKSPTEALLKLIAEAKGITETEAKEEAEKALKE

SEQ ID NO: 188 - protein, 99 aa, Synthetic Construct
GKSPTEALLKLIAEAKGITSTEAKEEAIKALKEGKSPTEALLKLIAEAKGITELEAKVLAEKALKEGKSPTEALLKLIAEAKGITETEAKLEAEKALKE

SEQ ID NO: 189 - protein, 99 aa, Synthetic Construct
GKSPTEVLLELIAEASGTTKEEVKEKFLKELSKGKSPTEVLLELIAEASGTTKEEVKEKFLKELSKGKSPTEVLLELIAEASGTTKEEVKEKFLKELSK

SEQ ID NO: 190 - protein, 99 aa, Synthetic Construct
GKSPTEVLLELIAEASGTTKEEVKRKFLKELSKGKSPTEVLLELIAEASGTTKAEVKREFLWELSLGKSPTEVLLELIAEASGTTKEEVKEKFLAELEK

SEQ ID NO: 191 - protein, 99 aa, Synthetic Construct
GKSPTEVLLELIAEASGTTREEVKEKFLKELRKGKSPTEVLLELIAEASGTTKEEVKEKFLKELSFGKSPTEVLLELIAEASGTTKEEVKKKFWKELSL

SEQ ID NO: 192 - protein, 99 aa, Synthetic Construct
GKSPTEVLLELIAEASGTTKEEVKEKFLKELSKGKSPTEVLLELIAEASGTTKEEVKEKFLKELSKGKSPTEVLLELIAEASGTTKREVKRWFLFELRK

SEQ ID NO: 193 - protein, 99 aa, Synthetic Construct
GKSPTEVLLELIAEASGTTKAEVKLKFLFELSFGKSPTEVLLELIAEASGTTKEEVKEKFLKELFKGKSPTEVLLELIAEASGTTKEEVKEKFLKELSK

SEQ ID NO: 194 - protein, 99 aa, Synthetic Construct
GYSTTEALLILIAEASGTTVEQQKQRFKELVKKGYSTTEALLILIAEASGTTVEQQKQRFKELVKKGYSTTEALLILIAEASGTTVEQQKQRFKELVKK

SEQ ID NO: 195 - protein, 198 aa, Synthetic Construct
GDKTAIAQILAIKASAKGDETELERALRYAKKVGDKTAIAQILAIKASAKGDETELERALRYAKKVGDKTAIAQILAIKASAKGDETELERALRYAKKVGDKTAIAQILAIKASAKGDETELERALRYAKKVGDKTAIAQILAIKASAKGDETELERALRYAKKVGDKTAIAQILAIKASAKGDETELERALRYAKKV

SEQ ID NO: 196 - protein, 198 aa, Synthetic Construct
GDKTAIAQILAIKASAKGDETELERALRYAVKVGDKTAIAQILAIKASAKGDETELEQALRYAKFVGDKTAIAQILAIKASAKGDELELTRALAYAKKVGDKTAIAQILAIKASAKGDETELERALRYAKLVGDKTAIAQILAIKASAKGDETELERALRYAKYVGDKTAIAQILAIKASAKGDEPELEYALAYAKKV

SEQ ID NO: 197 - protein, 198 aa, Synthetic Construct
GDKTAIAQILAIKASAKGDETELERALRYAKKVGDKTAIAQILAIKASAKGDETELERALIFAEAVGDKTAIAQILAIKASAKGDETELERALRYAKKVNDKTAIAQILAIKASAKGDETELDRALWYAKKVGDKTAIAQILAIKASAKGDETELERALRYAKKVGDKTAIAQILAIKASAKGDETELERALLYAKKV

SEQ ID NO: 198 - protein, 198 aa, Synthetic Construct
GDKTAIAQILAIKASAKGDETELERALRYAKKVGDKTAIAQILAIKASAKGDETELERALAYARLVGDKTAIAQILAIKASAKGDETELERALRYAEKVGDKTAIAQILAIKASAKGDEQELEAALIYAKKVGDKTAIAQILAIKASAKGDETELERALRYAKKVGDKTAIAQILAIKASAKGDETELERALWYAKKV

SEQ ID NO: 199 - protein, 198 aa, Synthetic Construct
GDRSAIATAYIALAEYLGDKEALLKAIEIAIKLGDRSAIAEAYIALARYLGDKEALLKAIEIAIKLGDRSAIATAYIALAEYLGDKEALLKAIEIAIKLGDRSAIAEAYIALARYLGDKEALLKAIEIAIKLGDRSAIATAYIALAEYLGDKEALLKAIEIAIKLGDRSAIAEAYIALARYLGDKEALLKAIEIAIKL

SEQ ID NO: 200 - protein, 198 aa, Synthetic Construct
GDKSALAQILAIYASAYGDTTLFLRALKLAKEVGDKSALAQILAIYASAYGDTTLFLRALKLAKEVGDKSALAQILAIYASAYGDTTLFLRALKLAKEVGDKSALAQILAIYASAYGDTTLFLRALKLAKEVGDKSALAQILAIYASAYGDTTLFLRALKLAKEVGDKSALAQILAIYASAYGDTTLFLRALKLAKEV

SEQ ID NO: 201 - protein, 198 aa, Synthetic Construct
GDLELYIRVLAIVAEAEGDKTKLELALKLALKKGDLKLYIEVLAIVAEAEGDKTKLELALKLALKKGDLELYIRVLAIVAKAEGDKTKLELALKLALKKGDLKLYIEVLAIVAEAEGDKTKLELALKLALKKGDLELYIRVLAIVAEAEGDKTKLELALKLALKKGDLKLYIEVLAIVAKAEGDKTKLELALKLALKK

SEQ ID NO: 202 - protein, 210 aa, Synthetic Construct
VSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLGVSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLG

SEQ ID NO: 203 - protein, 210 aa, Synthetic Construct
VSLEQALKILKVAAELGTTVEEAVKRALKLKTKLGVSLEQALKILEVAAELGTTVEEAVKRALKLKTKLGVSLEQALKILEVAAKLGTTVEEAVKRALKLKTKLGVSLEQALKILKVAAELGTTVEEAVKRALKLKTKLGVSLEQALKILEVAAELGTTVEEAVKRAMKLKTKLGVSLEQALKILEVAAKLGTTVEEAVKRALKLKTKLG

SEQ ID NO: 204 - protein, 279 aa, Synthetic Construct
GISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKL

SEQ ID NO: 205 - protein, 372 aa, Synthetic Construct
GISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKLGISVEELLKLAKAAYYSGTTVEEAYKLALKLGISVEELLKLAEAAYYSGTTVEEAYKLALKLGISVEELLKLAKAAYYSGTTVEEAYKLALKL

SEQ ID NO: 206 - protein, 13 aa, Synthetic Construct
PTEALLKLIAEAK

SEQ ID NO: 207 - protein, 14 aa, Synthetic Construct
ETEAKEEAEKALKE

SEQ ID NO: 208 - protein, 14 aa, Synthetic Construct
STEAKEEAIKALKE

SEQ ID NO: 209 - protein, 14 aa, Synthetic Construct
ELEAKVLAEKALKE

SEQ ID NO: 210 - protein, 14 aa, Synthetic Construct
ETEAKLEAEKALKE

SEQ ID NO: 211 - protein, 14 aa, Synthetic Construct
KEEVKEKFLKELSK

SEQ ID NO: 212 - protein, 14 aa, Synthetic Construct
KEEVKRKFLKELSK

SEQ ID NO: 213 - protein, 14 aa, Synthetic Construct
KAEVKREFLWELSL

SEQ ID NO: 214 - protein, 14 aa, Synthetic Construct
KEEVKEKFLAELEK

SEQ ID NO: 215 - protein, 14 aa, Synthetic Construct
KREVKRWFLFELRK

SEQ ID NO: 216 - protein, 14 aa, Synthetic Construct
KAEVKLKFLFELSF

SEQ ID NO: 217 - protein, 14 aa, Synthetic Construct
KEEVKEKFLKELFK

SEQ ID NO: 218 - protein, 13 aa, Synthetic Construct
TTEALLILIAEAS

SEQ ID NO: 219 - protein, 14 aa, Synthetic Construct
VEQQKQRFKELVKK

SEQ ID NO: 220 - protein, 13 aa, Synthetic Construct
TELERALRYAKKV

SEQ ID NO: 221 - protein, 13 aa, Synthetic Construct
TELERALIFAEAV

SEQ ID NO: 222 - protein, 13 aa, Synthetic Construct
TELDRALWYAKKV

SEQ ID NO: 223 - protein, 13 aa, Synthetic Construct
TELERALLYAKKV

SEQ ID NO: 224 - protein, 13 aa, Synthetic Construct
TELERALAYARLV

SEQ ID NO: 225 - protein, 13 aa, Synthetic Construct
TELERALRYAEKV

SEQ ID NO: 226 - protein, 13 aa, Synthetic Construct
TELERALWYAKKV

SEQ ID NO: 227 - protein, 14 aa, Synthetic Construct
SAIATAYIALAEYL

SEQ ID NO: 228 - protein, 13 aa, Synthetic Construct
EALLKAIEIAIKL

SEQ ID NO: 229 - protein, 14 aa, Synthetic Construct
SAIAEAYIALARYL

SEQ ID NO: 230 - protein, 14 aa, Synthetic Construct
SALAQILAIYASAY

SEQ ID NO: 231 - protein, 13 aa, Synthetic Construct
TLFLRALKLAKEV

SEQ ID NO: 232 - protein, 14 aa, Synthetic Construct
ELYIRVLAIVAEAE

SEQ ID NO: 233 - protein, 13 aa, Synthetic Construct
TKLELALKLALKK

SEQ ID NO: 234 - protein, 14 aa, Synthetic Construct
KLYIEVLAIVAEAE

SEQ ID NO: 235 - protein, 14 aa, Synthetic Construct
ELYIRVLAIVAKAE

SEQ ID NO: 236 - protein, 14 aa, Synthetic Construct
KLYIEVLAIVAKAE

SEQ ID NO: 237-238 - skipped (2 entries)

SEQ ID NO: 239 - protein, 33 aa, Synthetic Construct
GKSPTEALLKLIAEAKGITETEAKEEAEKALKE

SEQ ID NO: 240 - protein, 33 aa, Synthetic Construct
GKSPTEALLKLIAEAKGITSTEAKEEAIKALKE

SEQ ID NO: 241 - protein, 33 aa, Synthetic Construct
GKSPTEALLKLIAEAKGITELEAKVLAEKALKE

SEQ ID NO: 242 - protein, 33 aa, Synthetic Construct
GKSPTEALLKLIAEAKGITETEAKLEAEKALKE

SEQ ID NO: 243 - protein, 33 aa, Synthetic Construct
GKSPTEVLLELIAEASGTTKEEVKEKFLKELSK

SEQ ID NO: 244 - protein, 33 aa, Synthetic Construct
GKSPTEVLLELIAEASGTTKEEVKRKFLKELSK

SEQ ID NO: 245 - protein, 33 aa, Synthetic Construct
GKSPTEVLLELIAEASGTTKAEVKREFLWELSL

SEQ ID NO: 246 - protein, 33 aa, Synthetic Construct
GKSPTEVLLELIAEASGTTKEEVKEKFLAELEK

SEQ ID NO: 247 - protein, 33 aa, Synthetic Construct
GKSPTEVLLELIAEASGTTKREVKRWFLFELRK

SEQ ID NO: 248 - protein, 33 aa, Synthetic Construct
GKSPTEVLLELIAEASGTTKAEVKLKFLFELSF

SEQ ID NO: 249 - protein, 33 aa, Synthetic Construct
GKSPTEVLLELIAEASGTTKEEVKEKFLKELFK

SEQ ID NO: 250 - protein, 33 aa, Synthetic Construct
GYSTTEALLILIAEASGTTVEQQKQRFKELVKK

SEQ ID NO: 251 - protein, 33 aa, Synthetic Construct
GDKTAIAQILAIKASAKGDETELERALRYAKKV

SEQ ID NO: 252 - protein, 33 aa, Synthetic Construct
GDKTAIAQILAIKASAKGDETELERALIFAEAV

SEQ ID NO: 253 - protein, 33 aa, Synthetic Construct
NDKTAIAQILAIKASAKGDETELDRALWYAKKV

SEQ ID NO: 254 - protein, 33 aa, Synthetic Construct
GDKTAIAQILAIKASAKGDETELERALLYAKKV

SEQ ID NO: 255 - protein, 33 aa, Synthetic Construct
GDKTAIAQILAIKASAKGDETELERALAYARLV

SEQ ID NO: 256 - protein, 33 aa, Synthetic Construct
GDKTAIAQILAIKASAKGDETELERALRYAEKV

SEQ ID NO: 257 - protein, 33 aa, Synthetic Construct
GDKTAIAQILAIKASAKGDEQELEAALIYAKKV

SEQ ID NO: 258 - protein, 33 aa, Synthetic Construct
GDKTAIAQILAIKASAKGDETELERALWYAKKV

SEQ ID NO: 259 - protein, 33 aa, Synthetic Construct
GDRSAIATAYIALAEYLGDKEALLKAIEIAIKL

SEQ ID NO: 260 - protein, 33 aa, Synthetic Construct
GDRSAIAEAYIALARYLGDKEALLKAIEIAIKL

SEQ ID NO: 261 - protein, 33 aa, Synthetic Construct
GDKSALAQILAIYASAYGDTTLFLRALKLAKEV

SEQ ID NO: 262 - protein, 33 aa, Synthetic Construct
GDLELYIRVLAIVAEAEGDKTKLELALKLALKK

SEQ ID NO: 263 - protein, 33 aa, Synthetic Construct
GDLKLYIEVLAIVAEAEGDKTKLELALKLALKK

SEQ ID NO: 264 - protein, 33 aa, Synthetic Construct
GDLELYIRVLAIVAKAEGDKTKLELALKLALKK

SEQ ID NO: 265 - protein, 33 aa, Synthetic Construct
GDLKLYIEVLAIVAKAEGDKTKLELALKLALKK

SEQ ID NO: 266 - protein, 11 aa, Synthetic Construct
GGSGGGSGGSG

SEQ ID NO: 267 - protein, 10 aa, Synthetic Construct
GGSGGGSGSG

SEQ ID NO: 268 - protein, 8 aa, Synthetic Construct
GGSGGGSG

SEQ ID NO: 269 - protein, 10 aa, Synthetic Construct
GGGGSGGGGS

SEQ ID NO: 270 - protein, 8 aa, Synthetic Construct
GGGSGGGS

SEQ ID NO: 271 - protein, 6 aa, Synthetic Construct
GGSGGS

SEQ ID NO: 272 - protein, 15 aa, Synthetic Construct
GLNDIFEAQKIEWHE

SEQ ID NO: 273 - protein, 6 aa, Synthetic Construct
HHHHHH

SEQ ID NO: 274 - protein, 4 aa, Synthetic Construct
LPHD

SEQ ID NO: 275 - protein, 4 aa, Synthetic Construct
NPND

SEQ ID NO: 276 - protein, 4 aa, Synthetic Construct
DPKD

SEQ ID NO: 277 - protein, 5 aa, Synthetic Construct
GLEPD

SEQ ID NO: 278 - protein, 5 aa, Synthetic Construct
GVSLD

SEQ ID NO: 279 - protein, 5 aa, Synthetic Construct
GVLPD

SEQ ID NO: 280-283 - skipped (4 entries)

SEQ ID NO: 284 - protein, 20 aa, Synthetic Construct
METDTLLLWVLLLWVPGSTG

SEQ ID NO: 285 - protein, 5 aa, Synthetic Construct
DYKDE

SEQ ID NO: 286-289 - skipped (4 entries)

SEQ ID NO: 290 - protein, 16 aa, Synthetic Construct
GGGSGGGSGGGSGGGS

SEQ ID NO: 291 - protein, 263 aa, Synthetic Construct
CEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPEAIKAAAELGKAGISSEEILELLRAAHELGLDPECIKAAAELGKAGISSEEILELLRAAHELGL

SEQ ID NO: 292 - protein, 4 aa, Synthetic Construct
GGGS

SEQ ID NO: 293 - protein, 6 aa, Synthetic Construct
ENLYFQ